SEQUENCE LISTING (70 sequences)

SEQ ID NO: 1 - DNA, 801 bp, Artificial sequence
  gene hybrid OXA11/OXA7
atgaaaacatttgccgcatatgtaattatcgcgtgtctttcgagtacggcattagctagttcaattacagaaaatacgttttggaacaaagagttctctgccgaagccgtcaatggtgttttcgtgctttgtaaaagtagcagtaaatcctgcgctaccaataacttagctcgtgcatcaaaggaatatcttccagcatcaacatttaagatccccaacgcaattatcggcctagaaactggtgtcataaagaatgagcatcagattttcaaatgggacggaaaaccaagagccatgaaacaatgggaaagagacttgagcttaagaggggcaatacaagtttcagcggttcccgtatttcaacaaatcgccagagaagttggcgaagtaagaatgcagaaatatcttaaaaaattttcatatggtaaccagaatatcagtggcggcattgacaaattctggttggagggtcagcttagaatttccgcagttaatcaagtggagtttctagagtctctatttttaaataaattgtcagcatcaaaagaaaatcagctaatagtaaaagaggctttggtaacggaggctgcgcctgaatatcttgtgcattcaaaaactggtttttctggtgtgggaactgagtcaaatcctggtgtcgcatggtgggttggttgggttgagaagggagcagaggtttactttttcgcatttaacatggatatagacaacgaaaataagttgccgctaagaaaatccattcccaccaaaatcatggcaagtgagggcatcattggtggctaa

SEQ ID NO: 2 - protein, 266 aa, Artificial sequence
  protein hybrid OXA11/OXA7
MKTFAAYVIIACLSSTALASSITENTFWNKEFSAEAVNGVFVLCKSSSKSCATNNLARASKEYLPASTFKIPNAIIGLETGVIKNEHQIFKWDGKPRAMKQWERDLSLRGAIQVSAVPVFQQIAREVGEVRMQKYLKKFSYGNQNISGGIDKFWLEGQLRISAVNQVEFLESLFLNKLSASKENQLIVKEALVTEAAPEYLVHSKTGFSGVGTESNPGVAWWVGWVEKGAEVYFFAFNMDIDNENKLPLRKSIPTKIMASEGIIGG

SEQ ID NO: 3 - DNA, 801 bp, Artificial sequence
  gene hybrid OXO11/OXO7
atgaaaacatttgccgcatatgtaattatcgcgtgtctttcgagtacggcattagctggttcaattacagaaaatacgtcttggaacaaagagttctctgccgaagccgtcaatggtgtcttcgtgctttgtaaaagtagcagtaaatcctgcgctaccaatgacttagctcgtgcatcaaaggaatatcttccagcatcaacatttaagatccccaacgcaattatcggcctagaaactggtgtcataaagaatgagcatcagattttcaaatgggacggaaagccaagagccatgaaacaatgggaaagagacttgaccttaagaggggcaatacaagtttcagcggttcccgtatttcaacaaatcgccagagaagttggcgaagtaagaatgcagaaatatcttaaaaaattttcatatggtaaccagaatatcagtggtggcattgacaaattctggtcggagggtcagcttagaatttccgcagttaatcaagtggagtttctagagtctctatttttaaataaattgtcagcatcaaaagaaaatcagctaatagtaaaagaggctttggtaacggaggctgcgcctgaatatcttgtgcattcaaaaactggtttttctggtgtgggaactgagtcaaatcctggtgtcgcatggtgggttggttgggttgagaagggagcagaggtttactttttcgcatttaacatggatatagacaacgaaaataagttgccgctaagaaaatccattcccaccaaaatcatggcaagtgagggcatcattggtggctaa

SEQ ID NO: 4 - protein, 266 aa, Artificial sequence
  protein hybrid OXA11/OXA7
MKTFAAYVIIACLSSTALAGSITENTSWNKEFSAEAVNGVFVLCKSSSKSCATNDLARASKEYLPASTFKIPNAIIGLETGVIKNEHQIFKWDGKPRAMKQWERDLTLRGAIQVSAVPVFQQIAREVGEVRMQKYLKKFSYGNQNISGGIDKFWSEGQLRISAVNQVEFLESLFLNKLSASKENQLIVKEALVTEAAPEYLVHSKTGFSGVGTESNPGVAWWVGWVEKGAEVYFFAFNMDIDNENKLPLRKSIPTKIMASEGIIGG

SEQ ID NO: 5 - DNA, 801 bp, Artificial sequence
  gene hybrid OXA11/OXA7
atgaaaacatttgccgcatatgtaattatcgcgtgtctttcgagtacggcattagctggttcaattacagaaaatacgtcttggaacaaagagttctctgccgaagccgtcaatggtgtcttcgtgctttgtaaaagtagcagtaaatcctgcgctaccaatgacttagctcgtgcatcaaaggaatatcttccagcatcaacatttaagatccccaacgcaattatcggcctagaaactggtgtcataaagaatgagcatcaggttttcaaatgggacggaaagccaagagccatgaagcaatgggaaagagacttgaccttaagaggggcaatacaagtttcagctgttcccgtatttcaacaaatcgccagagaagttggcgaagtaagaatgcagaaataccttaaaaaattttcctatggcagccagaatatcagtggtggcattgacaaattctggttggaagaccagcttagaatttccgcagttaatcaagtggagtttctagagtctctatatttaaataaattgtcagcatctaaagaaaaccagctaatagtaaaagaggctttggtaacggaggcggcacctgaatatctagtgcattcaaaaactggtttttctggtgtgggaactgagtcaaatcctggtgtcgcatggtgggttgggtgggttgagaaggagacagaggtttactttttcgcctttaacatggatatagacaacgaaagtaagttgccgctaagaaaatccattcccaccaaaatcagggaaagtgagggcatcattggtggctaa

SEQ ID NO: 6 - protein, 266 aa, Artificial sequence
  protein hybrid OXA11/OXA7
MKTFAAYVIIACLSSTALAGSITENTSWNKEFSAEAVNGVFVLCKSSSKSCATNDLARASKEYLPASTFKIPNAIIGLETGVIKNEHQVFKWDGKPRAMKQWERDLTLRGAIQVSAVPVFQQIAREVGEVRMQKYLKKFSYGSQNISGGIDKFWLEDQLRISAVNQVEFLESLYLNKLSASKENQLIVKEALVTEAAPEYLVHSKTGFSGVGTESNPGVAWWVGWVEKETEVYFFAFNMDIDNESKLPLRKSIPTKIRESEGIIGG

SEQ ID NO: 7 - DNA, 801 bp, Artificial sequence
  gene hybrid OXA11/OXA7
atgaaaacatttgccgcatatgtaattatcgcgtgtctttcgagtacggcattagctggttcaattacagaaaatacgtcttggaacaaagagttctctgccgaagccgtcaatggtgtcttcgtgctttgtaaaagtagcagtaaatcctgcgctaccaatgacttagctcgtgcaccaaaggaatatcttccagcatcaacatttaagatccccaacgcaattatcggcctagaaactggtgtcataaagaatgagcatcaggttttcaaatgggacggaaagccaagagccatgaagcaatgggaaagagacttgaccttaagaggggcaatacaagtttcagctgttcccgtatttcaacaaatcgccagagaagttggcgaagtaagaatgcagaaataccttaaaaaattttcctatggcagccagaatatcagtggtggcattgacaaattctggttggaaggtcagcttagaatttccgcagttaatcaagtggagtttctagagtctctatttttaaataaattgtcagcatcaaaagaaaatcagctaatagtaaaagaggctttggtaacggaggctgcgcctgaatatcttgtgcattcaaaaactggtttttctggtgtgggaactgagtcaaatcctggtgtcgcatggtgggttggttgggttgagaagggagcagaggtttactttttcgcatttaacatggatatagacaacgaaaataagttgccgctaagaaaatccattcccaccaaaatcatggcaagtgagggcatcattggtggctaa

SEQ ID NO: 8 - protein, 266 aa, Artificial sequence
  protein hybrid OXA11/OXA7
MKTFAAYVIIACLSSTALAGSITENTSWNKEFSAEAVNGVFVLCKSSSKSCATNDLARAPKEYLPASTFKIPNAIIGLETGVIKNEHQVFKWDGKPRAMKQWERDLTLRGAIQVSAVPVFQQIAREVGEVRMQKYLKKFSYGSQNISGGIDKFWLEGQLRISAVNQVEFLESLFLNKLSASKENQLIVKEALVTEAAPEYLVHSKTGFSGVGTESNPGVAWWVGWVEKGAEVYFFAFNMDIDNENKLPLRKSIPTKIMASEGIIGG

SEQ ID NO: 9 - DNA, 801 bp, Artificial sequence
  gene hybrid OXA11/OXA7
atgaaaacatttgccgcatatgtaattatcgcgtgtctttcgagtacggcattagctggttcaattacagaaaatacgtcttggaacaaagagttctctgccgaagccgtcaatggtgtcttcgtgctttgtaaaagtagcagtaaatcctgcgctaccaatgacttagctcgtgcatcaaaggaatatcttccagcatcaacatttaagatccccaacgcaattatcggcctagaaactggtgtcataaagaatgagcatcaggttttcaaatgggacggaaagccaagagccatgaagcaatgggaaagagacttgaccttaagaggggcaatacaagtttcagctgttcccgtatttcaacaaatcgccagagaagttggcgaagtaagaatgcagaaataccttaaaaaattttcctatggcagccagaatatcagtggtggcattgacaaattctggttggaagaccagcttagaatttccgcagttaatcaagtggagtttctagagtctctatatttaaataaattgtcagcatctaaagaaaaccagctaatagtaaaagaggctttggtaacggaggcggcacctgaatatctagtgcattcaaaaactggtttttctggtgtgggaactgagtcaaatcctggtgtcgcatggtgggttgggtgggttgagaaggagacagaggtttactttttcgcctttaacatggatatggacaacgaaagtaagttgccgctaagaaaatccattcccaccaaaatcatggaaagtgagggcatcattggtggctaa

SEQ ID NO: 10 - protein, 266 aa, Artificial sequence
  protein hybrid OXA11/OXA7
MKTFAAYVIIACLSSTALAGSITENTSWNKEFSAEAVNGVFVLCKSSSKSCATNDLARASKEYLPASTFKIPNAIIGLETGVIKNEHQVFKWDGKPRAMKQWERDLTLRGAIQVSAVPVFQQIAREVGEVRMQKYLKKFSYGSQNISGGIDKFWLEDQLRISAVNQVEFLESLYLNKLSASKENQLIVKEALVTEAAPEYLVHSKTGFSGVGTESNPGVAWWVGWVEKETEVYFFAFNMDMDNESKLPLRKSIPTKIMESEGIIGG

SEQ ID NO: 11 - DNA, 801 bp, Artificial sequence
  gene hybrid OXA11/OXA7
atgaaaacatttgccgcatatgtaattactgcgtgtctttcaagtacggcattagctagttcaattacagaaaatacgttttggaacaaagagttctctgccgaagccgtcaatggtgttttcgtgctttgtaaaagtagcagtaaatcctgcgctacctataacttagctcgtgcatcaaaggaatatcttccagcatcaacatttaagatccccaacgcaattatcggcctagaaactggtgtcataaagaatgagcatcagattttcaaatgggacggaaagccaagagccatgaaacaatgggaaagagacttgagcttaagaggggcaatacaagtttcagcggttcccgtatttcaacaaatcgccagagaagttggcgaagtaagaatgcagaaatatcttaaaaaattttcatatggtaaccagaatatcagtggtggcattgacaaattctggttggagggtcagcttagaatttccgcagttaatcaagtggagtttctagagtctctatttttaaataaattgtcagcatcaaaagaaaatcagctaatagtaaaagaggctttggtaacggaggctgcgcctgaatatcttgtgcattcaaaaactggtttttctggtgtgggaactgagtcaaatcctggtgtcgcatggtgggttggttgggttgagaagggagcagaggtttactttttcgcatttaacatggatatagacaacgaaaataagttgccgctaagaaaatccattcccaccaaaatcatggcaagtgagggcatcattggtggctaa

SEQ ID NO: 12 - protein, 266 aa, Artificial sequence
  protein hybrid OXA11/OXA7
MKTFAAYVITACLSSTALASSITENTFWNKEFSAEAVNGVFVLCKSSSKSCATYNLARASKEYLPASTFKIPNAIIGLETGVIKNEHQIFKWDGKPRAMKQWERDLSLRGAIQVSAVPVFQQIAREVGEVRMQKYLKKFSYGNQNISGGIDKFWLEGQLRISAVNQVEFLESLFLNKLSASKENQLIVKEALVTEAAPEYLVHSKTGFSGVGTESNPGVAWWVGWVEKGAEVYFFAFNMDIDNENKLPLRKSIPTKIMASEGIIGG

SEQ ID NO: 13 - DNA, 801 bp, Artificial sequence
  gene hybrid OXA11/OXA7
atgaaaacatttgccgcatatgtaattatcgcgtgtctttcgagtacggcattagctggttcaattacagaaaatacgtcttggaacaaagagttctctgccgaagccgtcaatggtgtcttcgtgctttgtaaaagtagcagtaaatcctgcgctaccaatgacttagctcgtgcatcaaaggaatatcttccagcatcaacatttaagatccccaacgcaattatcggcctagaaactggtgtcataaggaatgagcatcaggttttcaaatgggacggaaagccaagagccatgaagcaatgggaaagagacttgaccttaagaggggcaatacaagtttcagctgttcccgtatttcaacaaatcgccagagaagttggcgaagtaagaatgcagaaataccttaaaaaattttcctatggcagccagaatatcagtggtggcattgacaaattctggttggaagaccagcttagaatttccgcagttaatcaagtggagtttctagagtctctatatttaaataaattgtcagcatctaaagaaaatcagctaatagtaaaagaggctttggtaacggaggctgcgcctgaatatcttgtgcattcaaaaactggtttttctggtgtgggaactgagtcaaatcctggtgtcgcatggtgggttgggtgggttgagaaggagacagaggtttactttttcgcatttaacatggatatagacaacgaaaataagttgccgctaagaaaattcattcccaccaaaatcatggcaagtgagggcatcattggtggctaa

SEQ ID NO: 14 - protein, 266 aa, Artificial sequence
  protein hybrid OXA11/OXA7
MKTFAAYVIIACLSSTALAGSITENTSWNKEFSAEAVNGVFVLCKSSSKSCATNDLARASKEYLPASTFKIPNAIIGLETGVIRNEHQVFKWDGKPRAMKQWERDLTLRGAIQVSAVPVFQQIAREVGEVRMQKYLKKFSYGSQNISGGIDKFWLEDQLRISAVNQVEFLESLYLNKLSASKENQLIVKEALVTEAAPEYLVHSKTGFSGVGTESNPGVAWWVGWVEKETEVYFFAFNMDIDNENKLPLRKFIPTKIMASEGIIGG

SEQ ID NO: 15 - DNA, 804 bp, Artificial sequence
  gene hybrid OXA11/OXA5
atgaaaacatttgccgcatatgtaattatcgcgtgtctttcgagtacggcattagctggttcaattacagaaaatacgtcttggaacaaagagttctctgccgaagccgtcaatggtgtcttcgtgctttgtaaaagtagcagtaaatcctgcgctaccaatgacttagctcgtgcatcaaaggaatatcttccagcatcaacatttaagatccccaacgcaattatcggcctagagactggtgtcataaagaatgagcatcaggttttcaaatgggacggaaagccaagagccatgaagcaatgggaaagagacttgaccttaagaggggcaatacaagtttcagctgttcccgtatttcaacaaatcgccagagaagttggcgaagtaagaatgcagaaataccttaaaaaattttcctatggcagccagaatatcagtggtggcattgacaaattctggttggaagaccagcttagaatttccgcagttaatcaagtggagtttctagagtctctatatttaaataaattgtcagcatctaaagaaaaccagctaatagtaaaagaggcaatagttacagaagcaactccagaatatatagttcattcaaaaactggtttttctggtgtgggaactgagtcaaatcctggtgtcgcatggtgggttgggtgggttgagaaggagacagaggtttactttttcgcctttaacatggatatagacaacgaaagtaagttgccgctaagaaaatccattcccaccaaaatcatggaaagtgagggcatcatcattggtggctaa

SEQ ID NO: 16 - protein, 267 aa, Artificial sequence
  protein hybrid OXA11/OXA5
MKTFAAYVIIACLSSTALAGSITENTSWNKEFSAEAVNGVFVLCKSSSKSCATNDLARASKEYLPASTFKIPNAIIGLETGVIKNEHQVFKWDGKPRAMKQWERDLTLRGAIQVSAVPVFQQIAREVGEVRMQKYLKKFSYGSQNISGGIDKFWLEDQLRISAVNQVEFLESLYLNKLSASKENQLIVKEAIVTEATPEYIVHSKTGFSGVGTESNPGVAWWVGWVEKETEVYFFAFNMDIDNESKLPLRKSIPTKIMESEGIIIGG

SEQ ID NO: 17 - DNA, 801 bp, Artificial sequence
  gene hybrid OXA11/OXA5
atgaaaacatttgccgcatatgtaattatcgcgtgtctttcgagtacggcattagctggttcaattacagaaaatacgtcttggaacaaagagttctctgccgaagccgtcaatggtgtcttcgtgctttgtaaaagtagcagtaaatcctgcgctaccaatgacttagctcgtgcatcaaaggaatatcttccagcatcaacatttaagatccccaacgcaattatcggcctagaaactggtgtcataaagaatgagcatcaggttttcaaatgggacggaaagccaagagccatgaagcaatgggaaagagacttgaccttaagaggggcaatacaagtttcagctgttcccgtatttcaacaaatcgccagagaagttggcgaagtaagaatgcagaaataccttaaaaaattttcctatggcagccagaatatcagtggtggcattgacaaattctggttggaagaccagcttagaatttccgcagttaatcaagtggagtttctagagtctctatatttaaataaattgtcagcatctaaagaaaaccagctaatagtaaaagaggctttggtaacggaggcggcacctgaatatctagtgcattcaaaaactggtttttctggtgtgggaactgagtcaaatcctggtgtcgcatggtgggttgggtgggtagagaaaggaactgaggtttactttttcgcctttagcatggatatagacaacgaaagtaagttgccgctaagaaaatccattcccaccaaaatcatggaaagtgagggcatcattggtggctaa

SEQ ID NO: 18 - protein, 266 aa, Artificial sequence
  protein hybrid OXA11/OXA7
MKTFAAYVIIACLSSTALAGSITENTSWNKEFSAEAVNGVFVLCKSSSKSCATNDLARASKEYLPASTFKIPNAIIGLETGVIKNEHQVFKWDGKPRAMKQWERDLTLRGAIQVSAVPVFQQIAREVGEVRMQKYLKKFSYGSQNISGGIDKFWLEDQLRISAVNQVEFLESLYLNKLSASKENQLIVKEALVTEAAPEYLVHSKTGFSGVGTESNPGVAWWVGWVEKGTEVYFFAFSMDIDNESKLPLRKSIPTKIMESEGIIGG

SEQ ID NO: 19 - DNA, 804 bp, Artificial sequence
  gene hybrid OXA11/OXA5
atgaaaacatttgccgcatatgtaattatcgcgtgtctttcgagtacggcattagctggttcaattacagaaaatacgtcttggaacaaagagttctctgccgaagccgtcaatggtgtcttcgtgctttgtaaaagtagcagtaaatcctgcgctaccaatgacttagctcgtgcatcaaaggaatatcttccagcatcaacatttaagatccccaacgcaattatcggcctagaaactggtgtcataaagaatgagcatcaggttttcaaatgggacggaaagccaagagccatgaagcaatgggaaagagacttgaccttaagaggggcaatacaagtttcagctgttcccgtatttcaacaaatcgccagagaagttggcgaagtaagaatgcagaaataccttaaaaaattttcctatggcagccagaatatcagtggtggcattgacaaattctggttggaagaccagcttagaatttccgcagttaatcaagtggagtttctagagtctctatatttaaataaattgtcagcatctaaagaaaaccagctaatagtaaaagaggctttggtaacggaggcggcacctgaatatctagtgcattcaaaaactggtttttctggtgtgggaactgagtcaaatcctggtgtcgcatggtgggttgggtgggttgagaaggagacagaggtttactttttcgcctttaacatggatatagacaacgagagtaaattgccgtcaagaaaatccatttcaacgaaaatcatggcaagtgaaggcatcatcattggtggctaa

SEQ ID NO: 20 - protein, 267 aa, Artificial sequence
  protein hybrid OXA11/OXA5
MKTFAAYVIIACLSSTALAGSITENTSWNKEFSAEAVNGVFVLCKSSSKSCATNDLARASKEYLPASTFKIPNAIIGLETGVIKNEHQVFKWDGKPRAMKQWERDLTLRGAIQVSAVPVFQQIAREVGEVRMQKYLKKFSYGSQNISGGIDKFWLEDQLRISAVNQVEFLESLYLNKLSASKENQLIVKEALVTEAAPEYLVHSKTGFSGVGTESNPGVAWWVGWVEKETEVYFFAFNMDIDNESKLPSRKSISTKIMASEGIIIGG

SEQ ID NO: 21 - DNA, 801 bp, Artificial sequence
  gene hybrid OXO11/OXO5
atgaaaacatttgccgcatatgtaattatcgcgtgtctttcgagtacggcattagctggttcaattacagaaaatacgtcttggaacaaagagttctctgccgaagccgtcaatggtgtcttcgtgctttgtaaaagtagcagtaaatcctgcgctaccaatgacttagctcgtgcatcaaaggaatatcttccagcatcaacatttaagatccccaacgcaattatcggcctagaaactggtgtcataaagaatgagcatcaggttttcaaatgggacggaaagccaagagccatgaagcaatgggaaagagacttgaccttaagaggggcaatacaagtttcagctgttcccgtatttcaacaaatcgccagagaagttggcgaagtgagaatgcagaaataccttaaaaaattttcctatggcagccagaatatcagtggtggcattgacaaattctggttggaagaccagcttagaatttccgcagttaatcaagtggagtctctagagtctctatatttaaataaattgtcagcatctaaagaaaaccagctaatagtaaaagaggctttggtaacggaggcggcacctgaatatctagtgcattcaaaaactggtttttctggtgtgggaactgagtcaaatcctggtgtcgcatggtgggttgggtgggttgagaaggagacagaggtttactttttcgcctttaacatggatatagacaacgaaagtaagttgccgctaagaaaatccattcccaccaaaatcatggaaagtgagggcatcattggtggctaa

SEQ ID NO: 22 - protein, 266 aa, Artificial sequence
  protein hybrid OXO11/OXO5
MKTFAAYVIIACLSSTALAGSITENTSWNKEFSAEAVNGVFVLCKSSSKSCATNDLARASKEYLPASTFKIPNAIIGLETGVIKNEHQVFKWDGKPRAMKQWERDLTLRGAIQVSAVPVFQQIAREVGEVRMQKYLKKFSYGSQNISGGIDKFWLEDQLRISAVNQVESLESLYLNKLSASKENQLIVKEALVTEAAPEYLVHSKTGFSGVGTESNPGVAWWVGWVEKETEVYFFAFNMDIDNESKLPLRKSIPTKIMESEGIIGG

SEQ ID NO: 23 - DNA, 804 bp, Artificial sequence
  gene hybrid OXO11/OXO5
atgaaaacatttgccgcatatgtaattatcgcgtgtctttcgagtacggcattagctggttcaattacagaaaatacgtcttggaacaaagagttctctgccgaagccgtcaatggtgtcttcgtgctttgtaaaagtagcagtaaatcctgcgctaccaatgacttagctcgtgcatcaaaggaatatcttccagcatcaacatttaagatccccaacgcaattatcggcctagaaactggtgtcataaagaatgagcatcaggttttcaaatgggacggaaagccaagagccatgaagcaatgggaaagagacttgaccttaagaggggcaatacaagtttcagctgttcccgtatttcaacaaatcgccagagaagttggcgaagtaagaatgcagaaataccttaaaaaattttcctatggcagccagaatatcagtggtggcattgacaaattctggttggaagaccagcttagaatttccgcagttaatcaagaggagtttctagagtctctatatttaaataaattgtcagcatctaaagaaaaccagctaatagtaaaagaggctttggtaacggaggcggcacctgaatatctagtgcattcaaaaactggtttttctggtgtgggaactgagtcaaatcctggtgtcgcatggtgggttgggtgggttgagaaggagacagaggtttactttttcgcctttaacatggatatagacaacgaaagtaagttgccgctaagaaaatccattcccaccaaaatcatggaaagtgagggcatcatcattggtggctaa

SEQ ID NO: 24 - protein, 267 aa, Artificial sequence
  protein hybrid OXO11/OXO5
MKTFAAYVIIACLSSTALAGSITENTSWNKEFSAEAVNGVFVLCKSSSKSCATNDLARASKEYLPASTFKIPNAIIGLETGVIKNEHQVFKWDGKPRAMKQWERDLTLRGAIQVSAVPVFQQIAREVGEVRMQKYLKKFSYGSQNISGGIDKFWLEDQLRISAVNQEEFLESLYLNKLSASKENQLIVKEALVTEAAPEYLVHSKTGFSGVGTESNPGVAWWVGWVEKETEVYFFAFNMDIDNESKLPLRKSIPTKIMESEGIIIGG

SEQ ID NO: 25 - DNA, 800 bp, Artificial sequence
  gene hybrid OXO11/OXO5
atgaaaacatttgccgcatatgtaattatcgcgtgtctttcgagtacggcattagctggttcaattacagaaaatacgtcttggaacaaagagttctctgccgaagccgtcaatggtgtcttcgtgctttgtaaaagtagcagtaaatcctgcgctaccaatgacttagctcgtgcatcaaaggaatatcttccagcatcaacatttaagatccccaacgcaattatcggcctagaaactggtgtcataaagaatgagcatcaggttttcaaatgggacggaaagccaagagccatgaagcaatgggaaagagacttgagcttaagaggggcaatacaagtttcagctgttcccgtatttcaacaaatcgccagagaagttggcgaagtaagaatgcagagataccttaaaaaattttcctatggcagccagaatatcagtggtggcattgacaaattctggttggaagaccagcttagaatttccgcagttaatcaagtggagtttctagagtctctatatttaaataaattgtcagcatctaaagaaaaccagctaatagtaaaagaggctttggtaacggaggcggcacctgaatatctagtgcattcaaaaactggtttttctggtgtgggaactgagtcaaatcctggtgtcgcatggtgggttgggtgggttgggaaggagacagaggtttactttttcgcctttaacatggatatagacaacgaaagtaagttgccgctaagaaatccattcccaccaaaatcatggaaagtgagggcatcattggtggctaa

SEQ ID NO: 26 - protein, 266 aa, Artificial sequence
  protein hybrid OXO11/OXO5
MKTFAAYVIIACLSSTALAGSITENTSWNKEFSAEAVNGVFVLCKSSSKSCATNDLARASKEYLPASTFKIPNAIIGLETGVIKNEHQVFKWDGKPRAMKQWERDLSLRGAIQVSAVPVFQQIAREVGEVRMQRYLKKFSYGSQNISGGIDKFWLEDQLRISAVNQVEFLESLYLNKLSASKENQLIVKEALVTEAAPEYLVHSKTGFSGVGTESNPGVAWWVGWVGKETEVYFFAFNMDIDNESKLPLRKSIPTKIMESEGIIGG

SEQ ID NO: 27 - DNA, 802 bp, Artificial sequence
  gene hybrid OXO11/OXO5
atgaaaacatttgccgcatatgtaattactgcgtgtctttcaagtacggcattagctagttcaattacagaaaatacgttttggaacaaagagttctctgccgaagccgtcaatggtgttttttcgtgctttgtaaaagtagcagtaaatcctgcgctaccaataacttagctcgtgcatcaaaggaatatcttccagcatcaacatttaagatccccaacgcaattatcggcctagaaactggtgtcataaagaatgagcatcaggttttcaaatgggacggaaagccaagagccatgaaacaatgggaaagagacttgagcttaagaggggcaatacaagtttcagcggttcccgtatttcaacaaatcgccagagaagttggcgaagtaagaatgcagaaatatcttaaaaaattttcatatggtaaccagaatatcagtggtggcattgacaaattctggttggagggtcagcttagaatttccgcagttaatcaagtggagtttctagagtctctatttttaaataaattgtcagcatcaaaagaaaatcagctaatagtaaaagaggctttggtaacggaggctgcgcctgaatatcttgtgcattcaaaaactggtttttctggtgtgggaactgagtcaaatcctggtgtcgcatggtgggttggttgggttgagaagggagcagaggtttactttttcgcatttaacatggatatagacaacgaaaataagttgccgctaagaaaatccattcccaccaaatcatggcaagtgagggcatcattggtggctaa

SEQ ID NO: 28 - protein, 266 aa, Artificial sequence
  protein hybrid OXO11/OXO5
MKTFAAYVITACLSSTALASSITENTFWNKEFSAEAVNGVFVLCKSSSKSCATNNLARASKEYLPASTFKIPNAIIGLETGVIKNEHQVFKWDGKPRAMKQWERDLSLRGAIQVSAVPVFQQIAREVGEVRMQKYLKKFSYGNQNISGGIDKFWLEGQLRISAVNQVEFLESLFLNKLSASKENQLIVKEALVTEAAPEYLVHSKTGFSGVGTESNPGVAWWVGWVEKGAEVYFFAFNMDIDNENKLPLRKSIPTKIMASEGIIGG

SEQ ID NO: 29 - DNA, 801 bp, Artificial sequence
  gene hybrid OXO11/OXO5
atgaaaacatttgccgcatacgtaattactgcgtgtctttcaagtacggcattagctagttcaattacagaaaatacgttttggaacaaagagttctctgccgaagccgtcaatggtgttttcgtgctttgtaaaagtagcagtaaatcctgcgctaccaatgacttagctcgtgcatcaaaggaatatcttccagcatcaacatttaagatccccaacgcaattatcggcctagaaactggtgtcataaagaatgagcatcagattttcaaatgggacggaaagccaagagccatgaagcaatgggaaagagacttgaccttaagaggggcaatacaagtttcagctgttcccgtatttcaacaaatcgccagagaagttggcgaagtaagaatgcagaaataccttaaaaaattttcatatggtaaccagaatatcagtggtggcattgacaaattctggttggagggtcagcttagaattcccgcagttaatcaagtggagtttctagagtctctatttttaaataaattgtcagcatcaaaagaaaatcagctaatagtaaaagaggctttggtaacggaggcggcacctgaatatcttgtgcattcaaaaactggtttttctggtgtgggaactgagtcaaatcctggtgtcgcatggtgggttggttgggttgagaagggagcagaggtttactttttcgcatttaacatggatatagacaacgaaaataagttgccgctaagaaaatccattcccaccaaaatcatggcaagtgagggcatcattggtggctaa

SEQ ID NO: 30 - protein, 266 aa, Artificial sequence
  protein hybrid OXO11/OXO5
MKTFAAYVITACLSSTALASSITENTFWNKEFSAEAVNGVFVLCKSSSKSCATNDLARASKEYLPASTFKIPNAIIGLETGVIKNEHQIFKWDGKPRAMKQWERDLTLRGAIQVSAVPVFQQIAREVGEVRMQKYLKKFSYGNQNISGGIDKFWLEGQLRIPAVNQVEFLESLFLNKLSASKENQLIVKEALVTEAAPEYLVHSKTGFSGVGTESNPGVAWWVGWVEKGAEVYFFAFNMDIDNENKLPLRKSIPTKIMASEGIIGG

SEQ ID NO: 31 - DNA, 801 bp, Artificial sequence
  gene hybrid OXO11/OXO5
atgaaaacatttgccgcatatgtaattatcgcgtgtctttcgagtacggcattagctggttcaattacagaaaatacgtcttggaacaaagagttctctgccgaagccgtcaatggtgtcttcgtgctttgtaaaagtagcagtaaatcctgcgctaccaatgacttagctcgtgcatcaaaggaatatcttccagcatcaacatttaagatccccaacgcaattatcggcctagaaactggtgtcataaagaatgagcatcaggttttcaaatgggacggaaagccaagagccatgaagcaatgggaaagagacttgaccttaagaggggcaatacaagtttcagctgttcccgtatttcaacaaatcgccagagaagttggcgaagtaagaatgcagaaataccttaaaaaattttcctatggcagccagaatatcagtggtggcattgacaaattctggttggaagaccagcttagaatttccgcagttaatcaagtggagtttctagagtctctatatttaaataaattgtcagcatctaaagaaaaccagctaatagtaaaagaggctttggtaacggaggcggcacctgaatatctagtgcattcaaaaactggtttttctggtgtgggaactgagtcaaatcctggtgtcgcatggtgggttgggtgggttgagaaggagacagaggtttactttttcgcctttaacatggatatagacaacgaaagtaagttgccgctaagaaaatccattcccaccaaaatcatggaaagtgagggcatcattggtggctaa

SEQ ID NO: 32 - protein, 266 aa, Artificial sequence
  protein hybrid OXO11/OXO5
MKTFAAYVIIACLSSTALAGSITENTSWNKEFSAEAVNGVFVLCKSSSKSCATNDLARASKEYLPASTFKIPNAIIGLETGVIKNEHQVFKWDGKPRAMKQWERDLTLRGAIQVSAVPVFQQIAREVGEVRMQKYLKKFSYGSQNISGGIDKFWLEDQLRISAVNQVEFLESLYLNKLSASKENQLIVKEALVTEAAPEYLVHSKTGFSGVGTESNPGVAWWVGWVEKETEVYFFAFNMDIDNESKLPLRKSIPTKIMESEGIIGG

SEQ ID NO: 33 - DNA, 801 bp, Artificial sequence
  gene hybrid OXO11/OXO5
atgaaaacatttgccgcatatgtaattatcgcgtgtctttcgagtacggcattagctggttcaattacagaaaatacgtcttggaacaaagagttctctgccgaagccgtcaatggtgtcttcgtgctttgtaaaagtagcagtaaatcctgcgctaccaatgacttagctcgtgcatcaaaggaatatcttccagcatcaacatttaagatccccaacgcaattatcggcctagaaactggtgtcataaagaatgagcatcaggttttcaaatgggacggaaagccaagagccatgaagcaatgggaaagagacttgaccttaagaggggcaatacaagtttcagctgttcccgtatttcaacaaatcgccagagaagttggcgaagtaagaatgcagaaataccttaaaaaattttcctatggcagccagaatatcagtggtggcattgacaaattctggttggaagaccagcttagaatttccgcagttaatcaagtggagtttctagagtctctatatttaaataaattgtcagcatctaaagaaaaccagctaatagtaaaagaggctttggtaacggaggcggcacctgaacatctagtgcattcaaaaactggtttttctggtgtgggaactgagtcaaatcctggtgtcgcatggtgggttgggtgggttgagaaggagacagaggtttactttttcgcctttaacatggacatagacaacgaaagtaagttgccgctaagaaaatccattcccaccaaaatcatggaaagtgagggcatcattggtggctaa

SEQ ID NO: 34 - protein, 266 aa, Artificial sequence
  protein hybrid OXO11/OXO5
MKTFAAYVIIACLSSTALAGSITENTSWNKEFSAEAVNGVFVLCKSSSKSCATNDLARASKEYLPASTFKIPNAIIGLETGVIKNEHQVFKWDGKPRAMKQWERDLTLRGAIQVSAVPVFQQIAREVGEVRMQKYLKKFSYGSQNISGGIDKFWLEDQLRISAVNQVEFLESLYLNKLSASKENQLIVKEALVTEAAPEHLVHSKTGFSGVGTESNPGVAWWVGWVEKETEVYFFAFNMDIDNESKLPLRKSIPTKIMESEGIIGG

SEQ ID NO: 35 - DNA, 804 bp, Artificial sequence
  gene hybrid OXO11/OXO5
atgaaaacatttgccgcatatttaattatcgcgtgtctttcgagtacggcattagctggttcaattacagaaaatacgtcttggaacaaagagttctctgccgaagccgtcaatggtgtcttcgtgctttgtaaaagtagcagtaaatcctgcgctaccaatgacttagctcgtgcatcaaaggaatatcttccagcatcaacatttaagatccccaacgcaattatcggcctagaaactggtgtcataaagaatgagcatcaggttttcaaatgggacggaaagccaagagccatgaagcaatgggaaagagacttgaccttaagaggggcaatacaagtttcagctgttcccgtatttcaacaaatcgccagagaagttggcgaagtaagaatgcagaaataccttaaaaaattttcctatggcagccagaatatcagtggtggcattgacaaattctggttggaagaccagcttagaatttccgcagttaatcaagtggagtttctagagtctctatatttaaataaattgtcagcatctaaagaaaaccagctaatagtaaaagaggctttggtaacggaggcggcacctgaatatctagtgcattcaaaaactggtttttctggtgtgggaactgagtcaaatcctggtgtcgcatggtgggttgggtgggttgagaaggagacagaggtttactttttcgcctttaacatggatatagacaacgaaagtaagttgccgctaagaaaatccattcccaccaaaatcatggaaagtgagggcatcatcattggtggctta

SEQ ID NO: 36 - protein, 267 aa, Artificial sequence
  protein hybrid OXO11/OXO5
MKTFAAYLIIACLSSTALAGSITENTSWNKEFSAEAVNGVFVLCKSSSKSCATNDLARASKEYLPASTFKIPNAIIGLETGVIKNEHQVFKWDGKPRAMKQWERDLTLRGAIQVSAVPVFQQIAREVGEVRMQKYLKKFSYGSQNISGGIDKFWLEDQLRISAVNQVEFLESLYLNKLSASKENQLIVKEALVTEAAPEYLVHSKTGFSGVGTESNPGVAWWVGWVEKETEVYFFAFNMDIDNESKLPLRKSIPTKIMESEGIIIGG

SEQ ID NO: 37 - DNA, 804 bp, Artificial sequence
  gene hybrid OXO11/OXO5
atgaaaacatttgccgcatatgtaattatcgcgtgtctttcgagtacggcattagctggttcaattacagaaaatacgtcttggaacaaagagttctctgccgaagccgtcaatggtgtcttcgtgctttgtaaaagtagcagtaaatcctgcgctaccaatgacttagctcgtgcatcaaaggaatatcttccagcatcaacatttaagatccccaacgcaattatcggcctagaaactggtgtcataaagaatgagcatcaggttttcaaatgggacggaaagccaagagccatgaagcaatgggaaagagacttgaccttaagaggggcaatacaagtttctgctgttcccgtatttcaacaaatcgccagagaagttggcgaagtaagaatgcagaaataccttaaaaaattttcctatggcagccagaatatcagtggtggcattgacaaattctggttggaagaccagcttagaatttccgcagttaatcaagtggagtttctagagtctctatatttaaataaattgtcagcatctaaagaaaaccagctaatagtaaaagaggctttggtaacggaggcggcacctgaatatctagtgcattcaaaaactggtttttctggtgtgggaactgagtcaaatcctggtgtcgcatggtgggttgggtgggttgagaaggagactgaggtttacttttttgcttctaacatggacatagacaatgagagtaaattgccgtcaagaaaatccatttcaacgaaaatcatggcaagtgaaggcatcatcattggtggctaa

SEQ ID NO: 38 - protein, 267 aa, Artificial sequence
  protein hybrid OXO11/OXO5
MKTFAAYVIIACLSSTALAGSITENTSWNKEFSAEAVNGVFVLCKSSSKSCATNDLARASKEYLPASTFKIPNAIIGLETGVIKNEHQVFKWDGKPRAMKQWERDLTLRGAIQVSAVPVFQQIAREVGEVRMQKYLKKFSYGSQNISGGIDKFWLEDQLRISAVNQVEFLESLYLNKLSASKENQLIVKEALVTEAAPEYLVHSKTGFSGVGTESNPGVAWWVGWVEKETEVYFFASNMDIDNESKLPSRKSISTKIMASEGIIIGG

SEQ ID NO: 39 - DNA, 801 bp, Pseudomonas aeruginosa
atgaaaacatttgccgcatatgtaattatcgcgtgtctttcgagtacggcattagctggttcaattacagaaaatacgtcttggaacaaagagttctctgccgaagccgtcaatggtgtcttcgtgctttgtaaaagtagcagtaaatcctgcgctaccaatgacttagctcgtgcatcaaaggaatatcttccagcatcaacatttaagatccccaacgcaattatcggcctagaaactggtgtcataaagaatgagcatcaggttttcaaatgggacggaaagccaagagccatgaagcaatgggaaagagacttgaccttaagaggggcaatacaagtttcagctgttcccgtatttcaacaaatcgccagagaagttggcgaagtaagaatgcagaaataccttaaaaaattttcctatggcagccagaatatcagtggtggcattgacaaatcctggttggaagaccagcttagaatttccgcagttaatcaagtggagtttctagagtctctatatttaaataaattgtcagcatctaaagaaaaccagctaatagtaaaagaggctttggtaacggaggcggcacctgaatatctagtgcattcaaaaactggtttttctggtgtgggaactgagtcaaatcctggtgtcgcatggtgggttgggtgggttgagaaggagacagaggtttactttttcgcctttaacatggatatagacaacgaaagtaagttgccgctaagaaaatccattcccaccaaaatcatggaaagtgagggcatcattggtggctaa

SEQ ID NO: 40 - DNA, 801 bp, Escherichia coli
atgaaaacatttgccgcatatgtaattactgcgtgtctttcaagtacggcattagctagttcaattacagaaaatacgttttggaacaaagagttctctgccgaagccgtcaatggtgttttcgtgctttgtaaaagtagcagtaaatcctgcgctaccaataacttagctcgtgcaccaaaggaatatcttccagcatcaacatttaagatccccaacgcaattatcggcctagaaactggtgtcataaagaatgagcatcagattttcaaatgggacggaaaaccaagagccatgaaacaatgggaaagagacttgagcttaagaggggcaatacaagtttcagcggttcccgtatttcaacaaatcgccagagaagttggcgaagtaagaatgcagaaatatcttaaaaaattttcatatggtaaccagaatatcagtggcggcattgacaaattctggtcggagggtcagcttagaatttccgcagttaatcaagtggagtttctagagtctctatttttaaataaattgtcagcatcaaaagaaaatcagctaatagtaaaagaggctttggtaacggaggctgcgcctgaatatcttgtgcattcaaaaactggtttttctggtgtgggaactgagtcaaatcctggtgtcgcatggtgggttggttgggttgagaagggagcagaggtttactttttcgcatttaacatggatatagacaacgaaaataagttgccgctaagaaaatccattcccaccaaaatcatggcaagtgagggcatcattggtggctaa

SEQ ID NO: 41 - DNA, 804 bp, Pseudomonas aeruginosa
atgaaaaccatagccgcatacttagttacttcctgtttttcaagcaccgcgctctcaaagtctatttctgaaaatttggtgtggaataaagaattttctagtgaatccgtacatggcgtttttgtactttgtaaaagtagtagcaattcctgtactacaaataatgcggcacgtgcatctacagcctatattccagcatcaacattcaaaattcctaatgctctaataggtcttgaaaccggcgccataaaagatgaacggcagattttcaaatgggacggcaagcccagagccatgaaacaatgggaaaaagacttaaggctaaggggcgctatacaggtttctgcggttccggtatttcaacaaattgccagagaagttggcgaaatgagaatgcaaagatatcttaacctgttttcatacggtaacgccaatatagggggaggcattgacaaattctggctagagggtcagcttagaatcccagcattcaatcaagataaatctttagagtcgctcttcctgaataatttgccagcatcaaaagcaaatcaactaatagtaaaagaggcaatagttacagaagctacgccagaatatattgttcattcaaaaactgggtattccggtgttggcacagaatcaagtcctggtgtcgcttggtgggttggttgggtagggaaaggagctgaggtttacttttttgcatttaacatggacatagacaatgagaataaattgccgtcaagaaaatccatttcaacgaaaatcatggcaagtgaaggcatcatcattggtggctaa

SEQ ID NO: 42 - DNA, 827 bp, Artificial sequence
  OUL3-05-II
atgaaaacatttgccgcatatgtaattatcgcgtgtctttcgagtacggcattagctggttcaattacagaaaatacgtcttggaacaaagagttctctgccgaagccgtcaatggtgtcttcgtgctttgtaaaagtagcagtaaatcctgcgctaccaatgacttagctcgtgcatcaaaggaatatcttccagcatcaacattcaaaattcctaatgctctaataggtcttgaaaccggcgccataaaagatgaacggcaggttttcaaatgggacggcaagcccagagccatgaagcaatgggaaaaagacttaaagctaaggggcgctatacaggtttctgctgttccggtatttcaacaaattgccagagaagttggcgaaataagaatgcaaaaataccttaacctgttttcatacggcaacgccaatatagggggaggcattgacaaattctggctagaaggtcagcttagaatctcagcattcaatcaagttaaatttttagagtcgctctacctgaataatttgccagcatcaaaagcaaaccaactaatagtaaaagaggcaatagttacagaagcaactccagaatatatagttcattcaaaaactgggtattccggtgttggcacagaatcaagtcctggtgtcgcttggtgggttggttgggtagagaaaggaactgaggtttacttttttgcttttaacatggacatagacaatgagagtaaattgccgtcaagaaaatccattcccaccaaaatcatggcaagtgagggcatcattggtggctaagctgtgaagatcccagcaaaggctta

SEQ ID NO: 43 - protein, 266 aa, Artificial sequence
  OUL3-05-II
MKTFAAYVIIACLSSTALAGSITENTSWNKEFSAEAVNGVFVLCKSSSKSCATNDLARASKEYLPASTFKIPNALIGLETGAIKDERQVFKWDGKPRAMKQWEKDLKLRGAIQVSAVPVFQQIAREVGEIRMQKYLNLFSYGNANIGGGIDKFWLEGQLRISAFNQVKFLESLYLNNLPASKANQLIVKEAIVTEATPEYIVHSKTGYSGVGTESSPGVAWWVGWVEKGTEVYFFAFNMDIDNESKLPSRKSIPTKIMASEGIIGG

SEQ ID NO: 44 - DNA, 801 bp, Artificial sequence
  OUL3-05-III
atgaaaacatttgccgcatatttagttctcgcgtgtctttcgagtacggcattagctggttcaattacagaaaatacgtcttggaacaaagagttctctgccgaagccgtcaatggtgtcttcgtgctttgtaaaagtagcagtaaatcctgcgctaccaatgacttagctcgtgcatcaaaggaatatcttccagcatcaacatttaagatccccaacgcaattatcggtctagaaactggtgtcataaagaatgagcatcaggttttcaaatgggacggaaagccaagagccatgaagcaatgggaaagagacttgaccttaagaggggcaatacaagtttcagctgttcccgtatttcaacaaatcgccagagaagttggcgaaataagaatgcagaaatatcttaaaaaattttcatatggtaaccagaatatcagtggtggcattgacaaattctggctagaaggtcagcttagaatctcagcattcaatcaagttaaatttttagagtcgctctacctgaataatttgccagcatcaaaagaaaatcagctaatagtaaaagaggctttggtaacggaggctgcgcctgaatatcttgtgcattcaaaaactggtttttctggtgtgggaactgagtcaaatcctggtgtcgcatggtgggttggttgggttgagaagggagcagaggtttactttttcgcatttaacatggatatagacaacgaaaataagttgccgctaagaaaatccattcccaccaaaatcatggcaagtgagggcatcattggtggctaa

SEQ ID NO: 45 - protein, 266 aa, Artificial sequence
  OUL3-05-III
MKTFAAYLVLACLSSTALAGSITENTSWNKEFSAEAVNGVFVLCKSSSKSCATNDLARASKEYLPASTFKIPNAIIGLETGVIKNEHQVFKWDGKPRAMKQWERDLTLRGAIQVSAVPVFQQIAREVGEIRMQKYLKKFSYGNQNISGGIDKFWLEGQLRISAFNQVKFLESLYLNNLPASKENQLIVKEALVTEAAPEYLVHSKTGFSGVGTESNPGVAWWVGWVEKGAEVYFFAFNMDIDNENKLPLRKSIPTKIMASEGIIGG

SEQ ID NO: 46 - DNA, 801 bp, Artificial sequence
  OUL3-05-IV
atgaaaacatttgccgcatatgtaattatcgcgtgtctttcgagtacggcattagctagttcaattacagaaaatacgtcttggaacaaagagttctctgccgaagccgtcaatggtgtcttcgtgctttgtaaaagtagcagtaaatcctgcgctaccaatgacttagctcgtgcatcaaaggaatatcttccagcatcaacatttaagatccccaacgcaattatcggcctagaaactggtgtcataaagaatgagcatcaggttttcaaatgggacggaaagccaagagccatgaagcaatgggaaagagacttgaccttaagaggggcaatacaagtttcagctgttcccgtatttcaacaaatcgccagagaagttggcgaaataagaatgcagaaatatcttaaaaaattttcatatggtaaccagaatatcagtggtggcattgacaaattctggttggagggtcagcttagaatttccgcagttaatcaagtggagtttctagagtctctatttttaaataaattgtcagcatcaaaagaaaatcagctaatagtaaaagaggctttggtaacggaggctgcgcctgaatatcttgtgcattcaaaaactggtttttctggtgtgggaactgagtcaaatcctggtgtcgcatggtgggttggttgggttgagaagggagcagaggtttactttttcgcatttaacatggatatagacaacgaaaataagttgccgctaagaaaatccattcccaccaaaatcatggcaagtgagggcatcattggtggctaa

SEQ ID NO: 47 - protein, 266 aa, Artificial Sequence
  OUL3_05_IV
MKTFAAYVIIACLSSTALASSITENTSWNKEFSAEAVNGVFVLCKSSSKSCATNDLARASKEYLPASTFKIPNAIIGLETGVIKNEHQVFKWDGKPRAMKQWERDLTLRGAIQVSAVPVFQQIAREVGEIRMQKYLKKFSYGNQNISGGIDKFWLEGQLRISAVNQVEFLESLFLNKLSASKENQLIVKEALVTEAAPEYLVHSKTGFSGVGTESNPGVAWWVGWVEKGAEVYFFAFNMDIDNENKLPLRKSIPTKIMASEGIIGG

SEQ ID NO: 48 - DNA, 801 bp, Artificial Sequence
  OUL3-05-IX
atgaaaacattagccgcatatttagttctagttttttatgcaagcaccgcgctctcagagtcaattacagaaaatttggcgtggaataaagaattttctagtgaatccgtacatggcgtttttgtactttgtaaaagtagtagcaattcctgtactacaaataatgcggcacgtgcatctacagcctatattccagcatcaacattcaaaattcctaatgctctaataggtcttgaaaccggcgccataaaagatgaacggcaggttttcaaatgggacggaaagccaagagccatgaagcaatgggaaagagacttaaagctaaggggcgctatacaggtttctgctgttccggtatttcaacaaattgccagagaagttggcgaaataagaatgcaaaaataccttaacctgttttcatacggcaacgccaatatagggggaggcattgacaaattctggctagaaggtcagcttagaatctcagcattcaatcaagttaaatttttagagtcgctctacctgaataaattgtcagcatcaaaagaaaaccaactaatagtaaaagaggcaatagttacagaagcaactccagaatatatagttcattcaaaaactggtttttctggtgttggcacagaatcaagtcctggtgtcgcttggtgggttggttgggtagagaaaggaactgaggtttacttttttgcttttaacatggacatagacaatgagagtaaattgccgtcaagaaaatccattcccaccaaaatcatggcaagtgagggcatcattggtggctaa

SEQ ID NO: 49 - protein, 266 aa, Artificial Sequence
  OUL3-05-IX
MKTLAAYLVLVFYASTALSESITENLAWNKEFSSESVHGVFVLCKSSSNSCTTNNAARASTAYIPASTFKIPNALIGLETGAIKDERQVFKWDGKPRAMKQWERDLKLRGAIQVSAVPVFQQIAREVGEIRMQKYLNLFSYGNANIGGGIDKFWLEGQLRISAFNQVKFLESLYLNKLSASKENQLIVKEAIVTEATPEYIVHSKTGFSGVGTESSPGVAWWVGWVEKGTEVYFFAFNMDIDNESKLPSRKSIPTKIMASEGIIGG

SEQ ID NO: 50 - DNA, 801 bp, Artificial Sequence
  OUL3-05-X
atgaaaacatttgccgcatatgtaattatcgcgtgtctttcgagtacggcattagctggttcaattacagaaaatacgtcttggaacaaagagttctctgccgaagccgtcaatggtgtcttcgtgctttgtaaaagtagcagtaaatcctgcgctaccaatgacttagctcgtgcatcaaaggaatatcttccagcatcaacatttaagatccccaacgcaattatcggcctagaaactggtgtcataaagaatgagcatcaggttttcaaatgggacggaaagccaagagccatgaagcaatgggaaagagacttgaccttaagaggggcaatacaagtttcagctgttcccgtatttcaacaaatcgccagagaagttggcgaagtaagaatgcagaaatatcttaaaaaattttcatatggtaaccagaatatcagtggtggcattgacaaattctggttggaaggtcagcttagaatttccgcagttaatcaagtggagtttctagagtctctatttttaaataaattgtcagcatcaaaagaaaatcagctaatagtaaaagaggctttggtaacggaggctgcgcctgaatatcttgtgcattcaaaaactggtttttctggtgtgggaactgagtcaaatcctggtgtcgcatggtgggttggttgggttgagaagggagcagaggtttactttttcgcatttaacatggatatagacaacgaaaataagttgccgctaagaaaatccattcccaccaaaatcatggcaagtgagggcatcattggtggctaa

SEQ ID NO: 51 - protein, 266 aa, Artificial Sequence
  OUL3-05-X
MKTFAAYVIIACLSSTALAGSITENTSWNKEFSAEAVNGVFVLCKSSSKSCATNDLARASKEYLPASTFKIPNAIIGLETGVIKNEHQVFKWDGKPRAMKQWERDLTLRGAIQVSAVPVFQQIAREVGEVRMQKYLKKFSYGNQNISGGIDKFWLEGQLRISAVNQVEFLESLFLNKLSASKENQLIVKEALVTEAAPEYLVHSKTGFSGVGTESNPGVAWWVGWVEKGAEVYFFAFNMDIDNENKLPLRKSIPTKIMASEGIIGG

SEQ ID NO: 52 - DNA, 1056 bp, Kluyveromyces
atgtctgctcacgaaatcccaaagacccagaaaggtgttatcttctacgagaccggtggtaagctggaatacaaggacatcgatgtcccaaccccaaaggccaacgagcttttggtcaacgtcaagtactccggtgtgtgccacactgacttgcacgcctaccacggtgactggccattgccagttaagttgcctctagtcggtggccacgagggtgccggtgtcgttgtcgccatgggtgagaacgtcaagggctggaaggtcggtgacttggccggtatcaagtggttgaacggctcctgtatgtcctgtgagtcctgtgagttgggtaacgagtccaactgtccagaggctgacttgtccggttacacccacgacggttctttccagcagtacgctactgccgatgccgtccaggccgctaagatcccagctggcgctgaccttgctgagatcgccccaatcctgtgtgccggtatcactgtctacaaggctttgaagtctgctaacttgcaggccggtgactgggttgccatctccggtgccgccggtggtttgggttccctagccgtccagtacgccaaggccatgggttaccgtgtcttgggtatcgacggtggtgaggagaaggagcagctcttcagacagttgggtggtgaggtcttcatcgacttcagaacctgcaaggacatcgagggtgagatcatcaaggccaccaacggtggtgctcacggtgtcatcaacgtctctgtctccgaggccgccatcgagtcctctaccaactacgtcagagccaacggtaccgtcgtcttggtcggtttgccagctggcgccaagtgcaagtctgacgttttcaaccaggtcgtcaagtccatctctatcgtcggttcttacgtcggtaacagagctgacaccagagaggctctagacttcttcgtccgtggtttggtcagatctccaatcaaggttgtcggtctatctactctaccagagattttcgagaagatggagaagggccaaattgttggcagatacgttgtcgacacctccaactaa

SEQ ID NO: 53 - DNA, 1045 bp, Saccharomyces
atgtctatcccagaaactcaaaaaggtgttatcttctacgaatcccacggtaagttggaatacaaagatattccagttccaaagccaaaggccaacgaattgttgatcaacgttaaatactctggtgtctgtcacactgacttgcacgcttggcacggtgactggccattgccagttaagctaccattagtcggtggtcacgaaggtgccggtgtcgttgtcggcatgggtgaaaacgttaagggctggaagatcggtgactacgccggtatcaaatggttgaacggttcttgtatggcctgtgaatactgtgaattgggtaacgaatccaactgtcctcacgctgacttgtctggttacacccacgacggttctttccaacaatacgctaccgctgacgctgttcaagccgctcacattcctcaaggtaccgacttggcccaagtcgcccccatcttgtgtgctggtatcaccgtctacaaggctttgaagtctgctaacttgatggccggtcactgggttgctatctccggtgctgctggtggtctaggttctttggctgttcaatacgccaaggctatgggttacagagtcttgggtattgacggtggtgaaggtaaggaagaattattcagatccatcggtggtgaagtcttcattgacttcactaaggaaaaggacattgtcggtgtgttctaaaggccactgacggtggtgctcacggtgtcatcaacgtttccgttccgaagccgctattgaagcttctaccagatacgttagagctaacggtaccaccgttttggtcggtatgccagctggtgccaagtgttgttctgatgtcttcaaccaagtcgtcaagtccatctctattgttggttcttacgtcggtaacagagctgacaccagagaagctttggacttcttcgccagaggtttggtcaagtctccaatcaaggttgtcggcttgtctaccttgccagaaatttacgaaaagatggaaaagggtcaaatcgttggtagatacgttgttgacacttctaaataa

SEQ ID NO: 54 - DNA, 1047 bp, Artificial sequence
  Clon A02
atgtctatcccagaaactcaaaaaggtgttatcttctacgaatcccacggtaagttggaatacaaagatattccagttccaaagccaaaggccaacgaattgttgatcaacgttaaatactctggtgtctgtcacactgacttgcacgcttggcacggtgactggccattgccagttaagctaccattagtcggtggtcacgaaggtgccggtgtcgttgtcggcatgggtgaaaacgttaagggctggaagatcggtgactacgccggtatcaaatggttgaacggttcttgtatggcctgtgaatactgtgaattgggtaacgaatccaactgtccagaggctgacttgtccggttacacccacgacggttctttccagcagtacgctactgccgatgccgtccaggccgctaagatcccagctggcgctgaccttgctgagatcgccccaatcctgtgtgccggtatcactgtctacaaggctttgaagtctgctaacttgcaggccggtgactgggttgccatctccggtgccgccggtggtttgggttccctagccgtccagtacgccaaggccatgggttaccgtgtcttgggtatcgacggtggtgaggagaaggagcagctcttcagacagttgggtggtgaggtcttcatcgacttcagaacctgcaaggacatcgagggtgagatcatcaaggccaccaacggtggcgctcacggtgtcatcaacgtctctgtctccgaggccgccatcgagtcctctaccaactacgtcagagccaacggtaccgtcgtcttggtcggtttgccagctggcgccaagtgcaagtctgacgttttcaaccaggtcgtcaagtccatctctatcgtcggttcttacgtcggtaacagagctgacaccagagaggctctagacttcttcgtccgtggtttggtcagatctccaatcaaggttgtcggtctatctactctaccagagattttcgagaagatggagaagggccaaattgttggcagatacgttgtcgacacctccaactaa

SEQ ID NO: 55 - DNA, 1060 bp, Artificial sequence
  Clon A03
gccatgggtgagaatgtctatcccagaaactcaaaaaggtgttatcttctacgaatcccacggtaagttggaatacaaagatattccagttccaaagccaaaggccaacgaattgttgatcaacgttaaatactctggtgtctgtcacactgacttgcacgcttggcacggtgactggccattgccagttaagctaccattagtcggtggtcacgaaggtgccggtgtcgttgtcgccatgggtgagaacgtcaagggctggaaggtcggtgacttggccggtatcaagtggttgaacggctcctgtatgtcctgtgagtcctgtgagttgggtaacgagtccaactgtccagaggctgacttgtccggttacacccacgacggttctttccagcagtacgctactgccgatgccgtccaggccgctaagatcccagctggcgctgaccttgctgagatcgccccaatcctgtgtgccggtatcactgtctacaaggctttgaagtctgctaacttgcaggccggtgactgggttgccatctccggtgccgccggtggtttgggttccctagccgtccagtacgccaaggccatgggttaccgtgtcttgggtatcgacggtggtgaggagaaggagcagctcttcagacagttgggtggtgaggtcttcatcgacttcagaacctgcaaggacatcgagggtgagatcatcaaggccaccaacggtggtgctcacggtgtcatcaacgtctctgtctccgaggccgccatcgagtcctctaccaactacgtcagagccaacggtaccgtcgtcttggtcggtttgccagctggcgccaagtgcaagtctgacgttttcaaccaggtcgtcaagtccatctctatcgtcggttcttacgtcggtaacagagctgacaccagagaggctctagacttcttcgtccgtggtttggtcagatctccaatcaaggttgtcggtctatctactctaccagagattttcgagaagatggagaagggccaaattgttggcagatacgttgtcgacacctccaactaa

SEQ ID NO: 56 - DNA, 1047 bp, Artificial sequence
  Clon A05
atgtctatcccaaagacccagaaaggtgttatcttctacgagaccggtggtaagctggaatacaaggacatcgatgtcccaaccccaaaggccaacgagcttttggtcaacgtcaagtactccggtgtgtgccacactgacttgcacgcctaccacggtgactggccattgccagttaagttgcctctagtcggtggccacgagggtgccggtgtcgttgtcgccatgggtgagaacgtcaagggctggaaggtcggtgacttggccggtatcaagtggttgaacggctcctgtatgtcctgtgagtcctgtgagttgggtaacgagtccaactgtccagaggctgacttgtccggttacacccacgacggttctttccagcagtacgctactgccgatgccgtccaggccgctaagatcccagctggcgctgaccttgctgagatcgccccaatcctgtgtgccggtatcactgtctacaaggctttgaagtctgctaacttgcaggccggtgactgggttgccatctccggtgccgccggtggtttgggttccctagccgtccagtacgccaaggccatgggttaccgtgtcttgggtatcgacggtggtgaggagaaggagcagctcttcagacagttgggtggtgaggtcttcatcgacttcagaacctgcaaggacatcgagggtgagatcatcaaggccaccaacggtggtgctcacggtgtcatcaacgtctctgtctccgaggccgccatcgagtcctctaccaactacgtcagagccaacggtaccgtcgtcttggtcggtttgccagctggcgccaagtgcaagtctgacgttttcaaccaggtcgtcaagtccatctctatcgtcggttcttacgtcggtaacagagctgacaccagagaggctctagacttcttcgtccgtggtttggtcagatctccaatcaaggttgtcggtctatctactctaccagagattttcgagaagatggagaagggccaaattgttggcagatacgttgtcgacacctccaactaa

SEQ ID NO: 57 - DNA, 1047 bp, Artificial sequence
  Clon A06
atgtctatcccagaaactcaaaaaggtgttatcttctacgaatcccacggtaagttggaatacaaagatattccagttccaaagccaaaggccaacgaattgttgatcaacgttaaatactctggtgtctgtcacactgacttgcacgcttggcacggtgactggccattgccagttaagctaccattagtcggtggtcacgaaggtgccggtgtcgttgtcggcatgggtgaaaacgttaagggctggaagatcggtgactacgccggtatcaaatggttgaacggttcttgtatggcctgtgaatactgtgaattgggtaacgagtccaactgtccagaggctgacttgtctggttacacccacgacggttctttccaacaatacgctactgccgatgccgtccaggccgctaagatcccagctggcgctgaccttgctgagatcgccccaatcctgtgtgccggtatcactgtctacaaggctttgaagtctgctaacttgcaggccggtgactgggttgccatctccggtgccgccggtggtttgggttccctagccgtccagtacgccaaggccatgggttaccgtgtcttgggtatcgacggtggtgaggagaaggagcagctcttcagacagttgggtggtgaggtcttcatcgacttcagaacctgcaaggacatcgagggtgagatcatcaaggccaccaacggtggtgctcacggtgtcatcaacgtctctgtctccgaggccgccatcgagtcctctaccaactacgtcagagccaacggtaccgtcgtcttggtcggtttgccagccggcgccaagtgcaagtctgacgttttcaaccaggtcgtcaagtccatctctatcgtcggttcttacgtcggtaacagagctgacaccagagaggctctagacttcttcgtccgtggtttggtcagatctccaatcaaggttgtcggtctatctactctaccagagattttcgagaagatggagaagggccaaattgttggcagatacgttgtcgacacctccaactaa

SEQ ID NO: 58 - DNA, 1049 bp, Artificial sequence
  Clon A10
atgtctatcccagaaactcaaaaaggtgttatcttctacgagaccggtggtaagctggaatacaaggacatcgatgtcccaaccccaaaggccaacgagcttttggtcaacgtcaagtactccggtgtgtgccacactgacttgcacgcctaccacggtgactggccattgccagttaagttgcctctagtcggtggccacgagggtgccggtgtcgttgtcgccatgggtgagaacgtcaagggctggaaggtcggtgacttggccggtatcaagtggttgaacggctcctgtatgtcctgtgagtcctgtgagttgggtaacgagtccaactgtccagaggctgacttgtccggttacacccacgacggttctttccagcagtacgctactgccgatgccgtccaggccgctaagatcccagctggcgctgaccttgctgagatcgccccaatcctgtgtgccggtatcactgtctacaaggctttgaagtctgctaacttgcaggccggtgactgggttgccatctccggtgccgccggtggtttgggttccctagccgtccagtacgccaaggccatgggtttaccgtgtcttgggtatcgacggtggtgaggagaaggagcagctcttcagacagttgggtggtggaggtcttcatcgacttcagaacctgcaaggacatcgagggtgagatcatcaagcccaccaacggtggtgctcacggtgtcatcaacgtctctgtctccgaggccgccatcgagtcctctaccaactacgtcagagccaacggtaccgtcgtcttggtcggtttgccagctggcgccaagtgcaagtctgacgttttcaaccaggtcgtcaagtccatctctatcgtcggttcttacgtcggtaacagagctgacaccagagaggctctagacttcttcgtccgtggtttggtcagatctccaatcaaggttgtcggtctatctactctaccagagattttcgagaagatggagaagggccaaattgttggcagatacgttgtcgacacctccaactaa

SEQ ID NO: 59 - DNA, 1047 bp, Artificial sequence
  Clon A11
atgtctatcccagaaactcaaaaaggtgttatcttctacgaatcccacggtaagttggaatacaaagatattccagttccaaagccaaaggccaacgaattgttgatcaacgttaaatactctggtgtctgtcacactgacttgcacgcttggcacggtgactggccattgccagttaagctaccattagtcggtggtcacgaaggtgccggtgtcgttgtcggcatgggtgaaaacgttaagggctggaagatcggtgactacgccggtatcaaatggttgaacggttcttgtatggcctgtgaatactgtgaattgggtaacgaatccaactgtcctcacgctgacttgtctggttacacccacgacggttctttccagcagtacgctactgccgatgccgtccaggccgctaagatcccagctggcgctgaccttgctgagatcgccccaatcctgtgtgccggtatcactgtctacaaggctttgaagtctgctaacttgcaggccggtgactgggttgccatctccggtgccgccggtggtttgggttccctagccgtccagtacgccaaggccatgggttaccgtgtcttgggtatcgacggtggtgaggagaaggagcagctcttcagacagttgggtggtgaggtcttcatcgacttcagaacctgcaaggacatcgagggtgagatcatcaaggccaccaacggtggtgctcacggtgtcatcaacgtctctgtctccgaggccgccatcgagtcctctaccaactacgtcagagccaacggtaccgtcgtcttggtcggtttgccagctggcgccaagtgcaagtctgacgttttcaaccaggtcgtcatgtccatctctatcgtcggttcttacgtcggtaacagagctgacaccagagaggctctagacttcttcgtccgtggtttggtcagatctccaatcaaggttgtcggtctatctactctaccagagattttcgagaagatggagaagggccaaattgttggcagatacgttgtcgacacctccaactaa

SEQ ID NO: 60 - DNA, 801 bp, Artificial Sequence
  gene hybrid
atgaaaaccatagccgcatattaagttctagtttttctttcgagtacggcattagctgagtcaattacagaaaatacgtcttggaacaaagagttctctgccgaagccgtcaatggtgtcttcgtgctttgtaaaagtagcagtaaatcctgcgctaccaatgacttagctcgtgcatcaaaggaatatcttccagcatcaacatttaagatccccaacgcaattatcggcctagaaactggtgtcataaagaatgagcatcaggttttcaaatgggacggaaagccaagagccatgaagcaatgggaaagagacttgaccttaagaggggcaatacaagtttcagctgttcccgtatttcaacaaattgccagagaagttggcgaaataagaatgcaaaaataccttaacctgttttcatccggcaacgccaatatagggggaggcattgacaaattctggctagaaggtcagcttagaatctcagcattcaatcaagttaaatttttagagtcgctctacctgaataatttgccagcatcaaaagcaaaccaactaatagtaaaagaggcaatagttacagaagcaactccagaatatatagttcattcaaaaactgggtattccggtgtgggaactgagtcaaatcctggtgtcgcatggtgggttgggtgggttgagaaggagacagaggtttactttttcgcctttaacatggatatagacaacgaaagtaagttgccgctaagaaaatccattcccaccaaaatcatggaaagtgagggcatcattggtggctaa

SEQ ID NO: 61 - protein, 265 aa, Artificial Sequence
  protein hybrid
MKTIAAYVLVFLSSTALAESITENTSWNKEFSAEAVNGVFVLCKSSSKSCATNDLARASKEYLPASTFKIPNAIIGLETGVIKNEHQVFKWDGKPRAMKQWERDLTLRGAIQVSAVPVFQQIAREVGEIRMQKYLNLFSSGNANIGGGIDKFWLEGQLRISAFNQVKFLESLYLNNLPASKANQLIVKEAIVTEATPEYIVHSKTGYSGVGTESNPGVAWWVGWVEKETEVYFFAFNMDIDNESKLPLRKSIPTKIMESEGIIGG

SEQ ID NO: 62 - DNA, 801 bp, Artificial Sequence
  gene hybrid
atgaaaacatttgccgcatatgtaattatcgcgtgtctttcgagtacggcattagctggatcaattacagaaaatacgtcttggaacaaagagttctctgcagaagccgtcaatggtgtcttcgtgctttgtaaaagtagcagtaaatcctgcgctaccaatgacacagctcgtgcatcaaaggaatatcttccagcatcaacattcaagatccccaacgcaattatcggcctagaaaccggcgccataaaagatgaacagcaggttttcaaatgggacggcaagcccagagccatgaagcaatgggaaaaagacttaagcctaaggggcgctatacaggtttctgctgttccggtatttcaacaaattgccagagaagttggcgaaatgagaatgcaaaaataccttaacctgttttcatacggcaacgccaatatagggggaggcattgacaaattctggctagaaggtcagcttagaatcccagcattcaatcaagttaaatttttagagtcgctctacctgaataatttgccagcatcaaaagcaacccacctaatagtaaaagaggcaatagtgacagaagcaactccagaatatatagttcattcaaaaactgggtattccggtgttggcacagaatcaagtcctggtgtcgcttggtgggttggttgggtagagaaaggaactgaggtttacttttttgcttttaacatggacatagacaatgagagtaaattgccgtcaagaaaatccatttcaacgaaaatcatggcaagtgaaggcatcattggtggctaa

SEQ ID NO: 63 - protein, 266 aa, Artificial Sequence
  protein hybrid
MKTFAAYVIIACLSSTALAGSITENTSWNKEFSAEAVNGVFVLCKSSSKSCATNDTARASKEYLPASTFKIPNAIIGLETGAIKDEQQVFKWDGKPRAMKQWEKDLSLRGAIQVSAVPVFQQIAREVGEMRMQKYLNLFSYGNANIGGGIDKFWLEGQLRIPAFNQVKFLESLYLNNLPASKATHLIVKEAIVTEATPEYIVHSKTGYSGVGTESSPGVAWWVGWVEKGTEVYFFAFNMDIDNESKLPSRKSISTKIMASEGIIGG

SEQ ID NO: 64 - DNA, 933 bp, Artificial Sequence
  gene hybrid
atgaaaacatttgccgcatatgtaattatcgcgtgtctttcgagtacggcattagctggttcaattacagaaaatacgtcttggaacaaagagttctctgccgaagccgtcaatggtgtcttcgtgctttgtaaaagtagcagtaaatcctgcgctaccaatgacttagctcgtgcatcaacagatatctctactgttgcatctccattatttgaaggaactgaaggttgttttttactttacgatgcatccacaaacgctgaaattgctcaattcaataaagcaaagtgtgcaacgcaaatggcaccagattcaactttcaagatcgcattatcacttatggtatttgatgcggaaataatagatcagaaaaccatattcaaatgggataaaaaccccaaaggaatggagatctggaacagcaatcatacaccaaagacgtggatgcaattttctgttgtttgggtttcgcaagaaataacccaaaaaattggattaaataaaatcaagaattatctcaaagattttgattatggaaatcaagacttctctggagataaagaaagaaacaacggattaacagaagcatggctcgaaagtagcttaaaaatttcaccagaagaacaaattcaattcctgcgtaaaattattaatcacaatctcccagttaaaaactcagccatagaaaacaccatagagaacatgtatctacaagatctggataatagtacaaaactgtatgggaaaactggtgcaggattcacagcaaatagaaccttacaaaacggatggtttgaagggtttattataagcaaatcagggcctaaatatgtttttgtgtccgcacttacaggaaacttggggttgaatttaacatcacccttaaaacccaagaagaatgcgatcccccttctaaacacactaaatttttaa

SEQ ID NO: 65 - protein, 310 aa, Artificial Sequence
  protein hybrid
MKTFAAYVIIACLSSTALAGSITENTSWNKEFSAEAVNGVFVLCKSSSKSCATNDLARASTDISTVASPLFEGTEGCFLLYDASTNAEIAQFNKAKCATQMAPDSTFKIALSLMVFDAEIIDQKTIFKWDKNPKGMEIWNSNHTPKTWMQFSVVWVSQEITQKIGLNKIKNYLKDFDYGNQDFSGDKERNNGLTEAWLESSLKISPEEQIQFLRKIINHNLPVKNSAIENTIENMYLQDLDNSTKLYGKTGAGFTANRTLQNGWFEGFIISKSGPKYVFVSALTGNLGLNLTSPLKPKKNAIPLLNTLNF

SEQ ID NO: 66 - DNA, 831 bp, Escherichia coli
atgaaaaacacaatacatatcaacttcgctatttttttaataattgcaaatattatctacagcagcgccagtgcatcaacagatatctctactgttgcatctccattatttgaaggaactgaaggttgttttttactttacgatgcatccacaaacgctgaaattgctcaattcaataaagcaaagtgtgcaacgcaaatggcaccagattcaactttcaagatcgcattatcacttatggtatttgatgcggaaataatagatcagaaaaccatattcaaatgggataaaacccccaaaggaatggagatctggaacagcaatcatacaccaaagacgtggatgcaattttctgttgtttgggtttcgcaagaaataacccaaaaaattggattaaataaaatcaagaattatctcaaagattttgattatggaaatcaagacttctctggagataaagaaagaaacaacggattaacagaagcatggctcgaaagtagcttaaaaatttcaccagaagaacaaattcaattcctgcgtaaaattattaatcacaatctcccagttaaaaactcagccatagaaaacaccatagagaacatgtatctacaagatctggataatagtacaaaactgtatgggaaaactggtgcaggattcacagcaaatagaaccttacaaaacggatggtttgaagggtttattataagcaaatcaggacataaatatgtttttgtgtccgcacttacaggaaacttggggtcgaatttaacatcaagcataaaagccaagaaaaatgcgatcaccattctaaacacactaaatttataa

SEQ ID NO: 67 - DNA, 1631 bp, Artificial sequence
  gene hybrid
atgaaaaacacaatacatatcaacttcgctatttttttaataattgcaaatattatctacagcagcgccagtgcatcaacagatatctctactgttgcatctccattatttgaaggaactgaaggttgttttttactttacgatgcatccacaaacgctgaaattgctcaattcaataaagcaaagtgtgcaacgcaaatggcaccagattcaactttcaagatcgcattatcacttatggtatttgatgcggaaataatagatcagaaaaccatattcaaatgggataaaacccccaaaggaatggagatctggaacagcaatcatacaccaaagacgtggatgcaattttctgttgtttgggtttcgcaagaaataacccaaaaaattggattaaataaaatcaagaattatctcaaagattttgattatggaaatcaagacttctctggagataaagaaagaaacaacggattaacagaagcatggctcgaaagtagcttaaaaatttcaccagaagaacaaattcaattcctgcgtaaaattattaatcacaatctcccagttaaaaactcagccatagaaaacaccatagagaacatgtatctacaagatctggataatagtacaaaactgtatgggaaaactggtgcaggattcacagcaaatagaaccttacaaaacggatggtttgaagggtttattataagcaaatcaggacataaatatgtttttgtgtccgcacttacaggaaacttggggtcgaatttaacatcaagcataaaagccaagaaaaatgcgatcaccattctaaacacactaaatttataatgaaaacatttgccgcatatgtaattatcgcgtgtctttcgagtacggcattagctggttcaattacagaaaatacgtcttggaacaaagagttctctgccgaagccgtcaatggtgtcttcgtgctttgtaaaagtagcagtaaatcctgcgctaccaatgacttagctcgtgcatcaaaggaatatcttccagcatcaacatttaagatccccaacgcaattatcggcctagaaactggtgtcataaagaatgagcatcaggttttcaaatgggacggaaagccaagagccatgaagcaatgggaaagagacttgaccttaagaggggcaatacaagtttcagctgttcccgtatttcaacaaatcgccagagaagttggcgaagtaagaatgcagaaataccttaaaaaattttcctatggcagccagaatatcagtggtggcattgacaaattctggttggaagaccagcttagaatttccgcagttaatcaagtggagtttctagagtctctatatttaaataaattgtcagcatctaaagaaaaccagctaatagtaaaagaggctttggtaacggaggcggcacctgaatatctagtgcattcaaaaactggtttttctggtgtgggaactgagtcaaatcctggtgtcgcatggtgggttgggtgggttgagaaggagacagaggtttactttttcgcctttaacatggatatagacaacgaaagtaagttgccgctaagaaaatccattcccaccaaaatcatggaaagtgagggcatcattggtggctaa

SEQ ID NO: 68 - protein, 276 aa, Artificial sequence
  hybrid
MKNTIHINFAIFLIIANIIYSSASASTDISTVASPLFEGTEGCFLLYDASTNAEIAQFNKAKCATQMAPDSTFKIALSLMVFDAEIIDQKTIFKWDKTPKGMEIWNSNHTPKTWMQFSVVWVSQEITQKIGLNKIKNYLKDFDYGNQDFSGDKERNNGLTEAWLESSLKISPEEQIQFLRKIINHNLPVKNSAIENTIENMYLQDLDNSTKLYGKTGAGFTANRTLQNGWFEGFIISKSGHKYVFVSALTGNLGSNLTSSIKAKKNAITILNTLNL

SEQ ID NO: 69 - DNA, 931 bp, Artificial sequence
  gene hybrid
atgaaaacatttgccgcatatgtaattatcgcgtgtctttcgagtacggcattagctggttcaattacagaaaatacgtcttggaacaaagagttctctgccgaagccgtcaatggtgtcttcgtgctttgtaaaagtagcagtaaatcctgcgctaccaatgacttagctgtgcatcaacgatatctctactgttgcatctccattatttgaaggaactgaaggttgttttttactttacgatgcatccacaaacgctgaaattgctcaattcaataaagcaaagtgtgcaacgcaaatggcaccagattcaactttcaagatcgcattatcacttatggtatttgatgcggaaataatagatcagaaaaccatattcaaatgggataaaaaccccaaaggaatggagatctggaacagcaatcatacaccaaagacgtggatgcaattttctgttgtttgggtttcgcaagaaataacccaaaaaattggattaaataaaatcaagaattatctcaaagattttgattatggaaatcaagacttctctggagataaagaaagaaacaacggattaacagaagcatggctcgaaagtagcttaaaaatttcaccagaagaacaaattcaattcctgcgtaaaattattaatcacaatctcccagttaaaaactcagccatagaaaacaccatagagaacatgtatctacaagatctggataatagtacaaaactgtatgggaaaactggtgcaggattcacagcaaatagaaccttacaaaacggatggtttgaagggtttattataagcaaatcagggactaaatatgtttttgtgtccgcacttacaggaaacttggggttgaatttaacatcacccttaaaacccaagaagaatgcgatcccccttctaaacacactaaatttttaa

SEQ ID NO: 70 - protein, 310 aa, Artificial sequence
  hybrid
MKTFAAYVIIACLSSTALAGSITENTSWNKEFSAEAVNGVFVLCKSSSKSCATNDLARASTDISTVASPLFEGTEGCFLLYDASTNAEIAQFNKAKCATQMAPDSTFKIALSLMVFDAEIIDQKTIFKWDKNPKGMEIWNSNHTPKTWMQFSVVWVSQEITQKIGLNKIKNYLKDFDYGNQDFSGDKERNNGLTEAWLESSLKISPEEQIQFLRKIINHNLPVKNSAIENTIENMYLQDLDNSTKLYGKTGAGFTANRTLQNGWFEGFIISKSGTKYVFVSALTGNLGLNLTSPLKPKKNAIPLLNTLNF

(19) United States
(12) Patent Application Publication
(10) Pub. No.: US 2014/0274803 A1
(43) Pub. Date: Sep. 18, 2014

(54) METHOD OF GENERATING GENE MOSAICS IN EUKARYOTIC CELLS

(71) Applicant: EVIAGENICS S.A., Villejuif (FR)

(72) Inventors: Rudy Pandjaitan, Maisons Alfort (FR); Alejandro Luque, Paris (FR)

(73) Assignee: Eviagenics S.A., Villejuif (FR)

(21) Appl. No.: 14/350,427

(22) PCT Filed: Oct. 12, 2012
(86) PCT No.: PCT/EP2012/070250

§ 371 Date: Apr. 8, 2014

(30) Foreign Application Priority Data
Oct. 12, 2011 (EP) 11184920.4

(51) Int. Cl. C12N 15/81; C12N 9/04

(52) U.S. Cl. CPC C12N 15/81; C12N 9/0006
USPC 506/14; 506/26; 435/254.2

(57) ABSTRACT

The invention relates to a method for generating a gene mosaic by somatic in vivo recombination, comprising a) in a single step procedure (i) transforming a cell with at least one gene A having a sequence homology of less than 99.5% to another gene to be recombined that is an integral part of the cell genome or presented in the framework of a genetic construct, (ii) recombining said genes, (iii) generating a gene mosaic of the genes at an integration site of a target genome, wherein said at least one gene A has a single flanking target sequence either at the 5′ end or 3′ end anchoring to the 5′ or 3′ end of said integration site, and b) selecting clones comprising the gene mosaic, wherein said cell is a eukaryotic strain with a knock-out of at least one DNA repair gene. The invention further refers to a method of producing a diversity of gene mosaics and gene assembly.

The invention refers to methods of generating gene mosaics by homeologous in vivo recombination in eukaryotic cells.

### BACKGROUND

One of the primary goals of protein design is to generate proteins with new or improved properties. The ability to confer a desired activity on a protein or enzyme has considerable practical application in the chemical and pharmaceutical industry. Directed protein evolution has emerged as a powerful technology platform in protein engineering, in which libraries of variants are searched experimentally for clones possessing the desired properties.

Directed protein evolution harnesses the power of natural selection to evolve proteins or nucleic acids with desirable properties not found in nature. Various techniques are used for generating protein mutants and variants and selecting desirable functions. Recombinant DNA technologies have allowed the transfer of single structural genes or genes for an entire pathway to a suitable surrogate host for rapid propagation and/or high-level protein production. Accumulated improvements in activity or other properties are usually obtained through iterations of mutation and screening. Applications of directed evolution are mainly found in academic and industrial laboratories to improve protein stability and enhance the activity or overall performance of enzymes and organisms or to alter enzyme substrate specificity and to design new activities. Most directed evolution projects seek to evolve properties that are useful to humans in an agricultural, medical or industrial context (biocatalysis).

The evolution of whole metabolic pathways is a particularly attractive concept, because most natural and novel compounds are produced by pathways rather than by single enzymes. Metabolic pathways engineering usually requires the coordinated manipulation of all enzymes in the pathway. The evolution of new metabolic pathways and the enhancement of bioprocessing is usually performed through a process of iterative cycles of recombination and screening or selection to evolve individual genes, whole plasmids, multigene clusters, or even whole genomes.

Shao et al (Nucleic Acids Research 37(2):e16 Epub 2008 Dec. 12) describe the assembly of large recombinant DNA encoding a whole biochemical pathway or genome in a single step via in vivo homologous recombination of two flanking (anchoring) regions at the 5′ and 3′ ends containing sequences of the 5′ or 3′ end of the adjacent fragment in Saccharomyces cerevisiae.

Elefanty et al. (Proc. Natl. Acad. Sci. 95, 11897-11902 (1998) describe gene targeting experiments to generate mutant mice, in which the lacZ reporter gene has been knocked into the SCL locus. Reference is made to FIG. 1 of their article showing the SCL-lacZ gene targeting strategy employing two anchoring sequences, i.e. one at each of the 5′ and 3′ end.

Directed evolution can be performed in living cells, also called in vivo evolution, or may not involve cells at all (in vitro evolution). In vivo evolution has the advantage of selecting for properties in a cellular environment, which is useful when the evolved protein or nucleic acid is to be used in living organisms. In vivo homologous recombination in yeast has been widely used for gene cloning, plasmid construction and library creation.

Library diversity is obtained through mutagenesis or recombination. DNA shuffling allows the direct recombination of beneficial mutations from multiple genes. In DNA shuffling a population of DNA sequences are randomly fragmented and then reassembled into full-length hybrid sequences.

For the purpose of homologous recombination naturally occurring homologous genes are used as the source of starting diversity. Single-gene shuffling library members are typically more than 95% identical. The family-shuffling, however, allows block exchanges of sequences that are typically more than 60% identical. The functional sequence diversity comes from related parental sequences that have survived natural selection; thus, much larger numbers of mutations are tolerated in a given sequence without introducing deleterious effects on the structure or function.

The recombination of DNA fragments of different origin with up to 30% diversity is described in WO1990007576A1. Hybrid genes are produced in vivo by intergeneric and/or interspecific recombination in mismatch repair deficient bacteria or in bacteria of which the mismatch repair (MMR) system is transitorily inactivated. Thereby those processes by which damaged DNA are repaired, are avoided, which would have an inhibitory effect on the recombination frequency between divergent sequences, i.e. homeologous recombination.

A review of basic mechanisms of MMR is provided by Kunz et al (Cell. Mol. Life Sci. 66 (2009) 1021-1038).

Targeted homeologous recombination is described in MMR deficient plants (WO2006/134496A2). Targeting to a locus with sequences having up to 10% differences was possible.

Homologous recombination into bacteria for the generation of polynucleotide libraries is disclosed in WO03/095658A1. An expression library of polynucleotides was generated, wherein each polynucleotide is integrated by homologous recombination into the genome of a competent bacterium host cell, using a non-replicating linear integration cassette comprising the polynucleotide and two flanking sequences homologous with a region of the host cell genome.

The diversity of libraries can be enhanced by taking advantage of the ability of haploid cells to efficiently mate leading to the formation of a diploid organism. In its vegetative life cycle S. cerevisiae cells have a haploid genome, i.e. every chromosome is present as a single copy. Under certain conditions the haploid cells can mate. By this way a diploid cell is formed. Diploid cells can form haploid cells again, especially when certain nutrients are missing. They then undergo a process called meiosis followed by sporulation to form four haploid spores. During meiosis the different chromosomes of the two parental genomes recombine. During meiotic recombination DNA fragments are exchanged resulting in recombined DNA material.

WO2005/075654A1 discloses a system for generating recombinant DNA sequences in Saccharomyces cerevisiae, which is based on the sexual reproductive cycle of S. cerevisiae. Heterozygous diploid cells are grown under conditions which induce the processes of meiosis and spore formation. Meiosis is generally characterized by elevated frequencies of genetic recombination. Thus, the products of meiosis, which are haploid cells or spores, can contain recombinant DNA sequences due to recombination between the two diverged DNA sequences. By an iterative method recombinant haploid progeny is selected and mated to one another, the resulting diploids are sporulated again, and their progeny spores are subjected to appropriate selection conditions to identify new recombination events. This process is described in wild-type or mismatch repair defective S. cerevisiae cells. Therefore, the genes of interest, each flanked by two selection markers, are integrated into an identical locus of each of the two sister chromosomes of mismatch repair deficient diploid strains. DNA sequences are added to the 5′ or 3′ end of the new DNA fragment that are 100% identical to the flanking DNA sequences of the locus where the DNA has to be integrated. These flanking target sequences are about 400-450 nucleotides long. Then the cells enter meiotic cycle and are forced to initiate sporulation. During the sporulation the recombination process takes place. The resulting spores and recombinant sequences can be differentiated by selection for the appropriate flanking markers.

The ability of yeast to efficiently recombine homologous DNA sequences can also be exploited to increase the diversity of a library. When two genes that share 89.9% homology were mutated by PCR and transformed into wild type yeast, a chimeric library of 10e7 was created through in vivo homologous recombination, showing several cross-over points throughout the two genes (Swers et al Nucleic Acids Research 32(3) e36 (2004)).

A method of mitotic homeologous recombination is described by Nicholson et al (Genetics 154: 133-146 (2000)). Effects of defined mismatches contained in short inverted repeats on recombination rates in wild-type or MMR-defective strains were investigated.

It is the object of the present invention to provide an improved method of preparing and assembling a diversity of gene mosaics, especially for recombining long DNA fragments. As a result it would be desirable to provide respective libraries of variants for the selection of improved recombinants.

The object is achieved by the provision of the embodiments of the present application.

### SUMMARY OF THE INVENTION

The present invention provides a novel method for generating a gene mosaic by somatic in vivo recombination, comprising
a) in a single step procedure
   (i) transforming a cell with at least one gene A having a sequence homology of less than 99.5% to another gene to be recombined that is an integral part of the cell genome or presented in the framework of a genetic construct, preferably employing at least one gene B to be recombined,
   (ii) recombining said genes,
   (iii) generating a gene mosaic of the genes at an integration site of a target genome, wherein said at least one gene A has a single flanking target sequence either at the 5′ end or 3′ end anchoring to the 5′ or 3′ end of said integration site, and
b) selecting clones comprising the gene mosaic,

wherein said cell is a eukaryotic strain with a knock-out of at least one DNA repair gene. When gene A is to be recombined with at least one gene B, the single flanking target sequence of gene A is preferably anchoring to the 5′ end of the integration site while gene B has a single flanking target sequence anchoring to the 3′ end.

Specifically the eukaryotic strain is a viable strain with a knock-out of at least one DNA repair gene.

Specifically said DNA repair gene is a gene involved in DNA repair mechanisms, such as a gene of the MSH/Mut, RecQ and RAD families.

According to a specific aspect the DNA repair gene is completely or temporarily knocked-out, preferably by mutation, such as deletion and/or insertion and/or substitution of one or more nucleotides.

The term “knock-out” as understood herein shall refer to any type of impairment of DNA structure and/or function. Such impairment may be through mutations of at least one DNA repair gene, e.g. by deletion of a gene of DNA repair, or by engineering mutants reducing its function. Alternative methods may refer to inactivate or inhibit such DNA repair by addition of respective agents, or overexpressing other genes, or by circumventing expressing said genes or their function. Such knock-out may be completely, e.g. losing the functional DNA repair, partly or temporarily, including reversible knock-out. Knock-out strains are specifically understood herein to be of strains with a knock-out of at least one DNA repair gene.

DNA repair genes are typically genes supporting the DNA repair process in a cell and actively responding to damage in the DNA structure. Depending on the type of damage inflicted on the DNA's double helical structure, there is a variety of repair strategies to restore lost information. If possible, cells use the unmodified complementary strand of the DNA or the sister chromatid as a template to recover the original information. Without access to a template, cells use an error-prone recovery mechanism known as translation synthesis. Damage to DNA alters the spatial configuration of the helix, and such alterations can be detected by the cell. Once damage is localized, specific DNA repair molecules bind at or near the site of damage, inducing other molecules to bind and form a complex that enables the actual repair to take place. Loss of DNA repair gene function would lead to a breakdown in the maintenance of genome integrity.

Examples of such DNA repair genes suitably knocked-out in the eukaryotic strains as used according to the invention, are helicases, such as the RecQ homologues or RecQ family of helicases in eukaryotic species, among them Sgs1 in the budding yeast, e.g. Saccharomyces cerevisiae, and Rqh1 in the fission yeast, e.g. Schizosaccharomyces pombe. Further examples are genes involved in nucleotide excision repair, e.g. the RAD homologues or RAD gene family in eukaryotic species.

Such DNA repair genes are understood to be different from the specific genes of DNA-mismatch correction, such as MutS or MutL. Hence the eukaryotic cells as used according to the invention are specifically no mismatch repair deficient cells.

Specifically preferred knock-outs are by deletion or mutations of genes that are essential for DNA repair, in particular deletion or mutation of a gene of the RAD or RECQ family, e.g. RAD1 and/or RECQ homologues in eukaryotic cells. According to a specific embodiment of the invention said DNA repair gene is selected from the group consisting of homologues or analogs of RAD1 and RECQ.

Preferred embodiments refer to knock-out strains selected from the group consisting of fungal, yeast, plant, insect and mammalian cells.

Specifically preferred are strains selected from the group consisting of Saccharomyces, Schizosaccharomyces, Saccharomyces, Candida, Kluyveromyces, Hansenula, Schizosaccaromyces, Yarrowia, Pichia, Aspergillus, Drosophila and Caenorhabditis.

Preferably haploid strains, such as haploid yeast strains are employed.

Alternatively, mammalian cells, like HeLa cells or Jurkat cells, or plant cells, like Arabidopsis, may be used.

Preferred strains are e.g. selected from group consisting of Saccharomyces cerevisiae with a knock-out of at least the SGS1 gene, Schizosaccharomyces pombe with a knock-out of at least the RQH1 gene, Drosophila melanogaster with a knock-out of at least the dmblm gene, Caenorhabditis elegans with a knock-out of at least one of F18C5.2 and T04A11.6 genes, plants with a knock-out of at least one of AtRECQL1 to 4 and 4B genes, and mammalian cells with a knock-out of at least one of BLM, WRN, RECQL, RECQL4 and RECQL5 genes.

Specifically the invention relates to a method for generating a gene mosaic by somatic in vivo recombination, comprising
a) in a single step procedure
   (i) transforming a cell with at least one gene A having a sequence homology of less than 99.5% to a different gene B which is an integral part of the cell genome or presented in the framework of a genetic construct or expression cassette,
   (ii) recombining said genes,
   (iii) generating a gene mosaic of genes A and B at an integration site of a target genome, wherein said at least one gene A is linked to a single flanking target sequence either at the 5′ end or 3′ end of the genetic construct anchoring to the 5′ or 3′ end of said integration site and
b) selecting clones comprising the gene mosaic,

wherein said cell is a eukaryotic strain with a knock-out of at least one DNA repair gene.

It is specifically preferred that a selection marker is used in the gene mosaic and the clones are selected according to the presence of the selection marker. For example, the gene mosaic comprises a selection marker, e.g. where said gene A is linked to a selection marker. Alternatively, selection may also be made by the presence of any product resulting of recombinants, e.g. through determining the yield or functional characteristics. Specifically one or more different selection markers may be used to differentiate the type of gene mosaics.

Specifically the method according to the invention employs said another gene that is part of the target genome, e.g. the genome of the cell. In a preferred embodiment said anther gene is gene B being part of the genome of the cell.

According to an alternatively preferred embodiment, said another gene is a genetic construct separate from the target genome, such as a linear polynucleotide, and optionally integrated into the target genome in the course of the recombination.

According to a specific embodiment of the invention the cell is co-transformed with at least one gene A and at least one gene B, wherein said single flanking target sequence of gene A is anchoring to the 5′ end of an integration site on said target genome, and wherein gene B is linked to a single flanking target sequence anchoring to the 3′ end of the integration site.

Specifically, the cell can be co-transformed with at least one gene A with a selection marker and at least one gene B, wherein said single flanking target sequence of gene A is anchoring to the 5′ end of an integration site on said target genome, and wherein gene B is linked to a different selection marker and a single flanking target sequence anchoring to the 3′ end of the integration site, and wherein clones for the at least two selection markers are selected.

Specifically, the cell can be co-transformed with at least two different genes A1 and A2 and optionally with at least two different genes B1 and B2.

According to a specific embodiment, at least one further gene C is co-transformed, which has a sequence hybridizing with a sequence of gene A and/or said another gene to obtain assembly of said further gene C to gene A and/or said another gene, preferably wherein at least one of the assembled genes has an intragenic gene mosaic.

Specifically, at least one further gene C is co-transformed, which has a sequence hybridizing with a sequence of gene A and/or B, e.g. the full length gene A or gene B or a partial sequence of gene A and/or B, to obtain recombination and assembly of said further gene C to gene A and/or B.

Specifically, the hybridizing sequence of said gene C has a sequence homology of less than 99.5% to said sequence, and preferably at least 30% sequence homology.

Specifically gene mosaics having at least one nucleotide exchange or cross-over within the genes are selected, i.e. mosaics with an intragenic cross-over, such as those comprising parts of gene A and parts of said another gene(s) combined, which is understood as a mixture of partial genes to obtain a recombined intragenic gene mosaic, such as genes suitable for the expression of products in a different way, e.g. having improved properties or at improved yields. Such intragenic gene mosaics can be produced by recombination and preferably also assembly of a series of genes, wherein one or more of the assembled genes have such intragenic gene mosaics.

According to a preferred embodiment, mosaics of at least three different genes A and/or B and/or C can be obtained.

Preferably, said gene A and/or said another gene is coding for a polypeptide or part of a polypeptide having an activity.

Specifically, the inventive method employs genes A, B and/or C which are coding for part of a polypeptide having an activity. Accordingly, the genes, such as genes A and/or B and/or C, preferably all of them do not individually encode a biologically active polypeptide as such, but would encode only part of it, and may bring about a respective activity or modified activity upon gene assembly only.

Using the inventive method, multiple genes coding for polypeptides of a biochemical pathway can be assembled and recombined.

In another specific embodiment, the inventive method provides for recombination and eventual assembly of genes resulting in a non-coding sequence, such as a promoter, untranslated region, ribosomal binding site, terminator, etc.

Any recombination competent eukaryotic host cell can be used for generating a gene mosaic by somatic in vivo recombination according to the present invention.

According to a specific embodiment, the flanking target sequence is at least 5 bp, preferably at least 10 bp, more preferably at least 20 bp, 50 bp, 100 bp up to 5,000 bp length. Specifically the flanking target sequence is linked to said gene or is an integral, terminal part of said gene. It is preferred that said the flanking target sequence has homology or corresponding sequence identity in the range of 30% to 99.5%, preferably less than 95%, less than 90%, less than 80%, even less than 70% or less than 60%, hybridising with the anchoring sequence of said integration site. It turned out that the method according to the invention provides for the efficient gene mosaic formation and library formation with a homology or corresponding sequence identity of even less than 50%, such as for example a homology of 47%, i.e. a diversity of 53%. Preferably the homology is at least 35 or 40%.

When at least two different flanking target sequences anchoring to the target integration site of the genome are used according to the invention, it is preferred that they do not recombine with each other, preferably they share less than 30% homology.

Selection markers useful for the inventive method can be selected from the group consisting of any of the known nutrition auxotrophic markers, antibiotics resistance markers, fluorescent markers, knock-in markers, activator/binding domain markers and dominant recessive markers and colorimetric markers. Preferred markers can be temporally inactivated or functionally knocked out, and may be re-established to regain its marking property. Further preferred markers are traceable genes, wherein the marker is a function of either of the gene sequences A and/or the other gene(s), such as gene B, without separate sequences with a marker function, so that the expression of the gene mosaic can be directly determined through detection of the mosaic itself. In this case the gene mosaic is directly traceable.

According to a specific embodiment, said genes are comprised in a linear polynucleotide, a vector or a yeast artificial chromosome. Specifically, gene A and/or other genes to be recombined are in the form of linear polynucleotides, preferably of 300 to 20.000 bp. Specifically, there would be no need to construct or employ plasmids or megaplasmids. The gene(s) can thus be used as such, i.e. without carrier.

The genes used for recombination and integration can also be comprised in any genetic construct, e.g. to be used as vector for carrying said gene(s). Said genes can thus be comprised in a genetic construct, e.g. a linear polynucleotide, a vector or a yeast artificial chromosome. These preferably include linear polynucleotides, plasmids, PCR constructs, artificial chromosomes, like yeast artificial chromosomes, viral vectors or transposable elements.

According to a specific embodiment of the invention the integration site of the target genome is located on either of the genes, e.g. within a linear polynucleotide, a plasmid or chromosome, including artificial chromosomes.

The method according to the invention specifically provides for the selection of at least one clone having an intragenic gene mosaic. Specifically, at least one clone having a gene assembly and at least one intragenic gene mosaic is selected.

Using the method according to the invention gene mosaics of at least 3, preferably at least 9, up to 20,000 base pairs can be obtained, as well as gene mosaics, e.g. comprising at least one intragenic mosaic, preferably with at least 3 cross-over events, preferably at least 4, 5, or 10 cross-over events per 700 base pairs, more preferably per 600 bp, per 500 bp or even below. Typically a high degree of cross-over events provides for a large diversity of recombined genes, which may be used to produce a library for selecting suitable library members. The degree of mosaics or cross-over events can be understood as a quality parameter of such a library.

The genes which are modified according to the method of the invention can be any genes useful for scientific or industrial purposes. These genes can be for example non-coding sequences, e.g. those which may be used for recombinant expression systems, or variants of polypeptides, in whole or in part, including those partial sequences, which do not encode a polypeptide with biological activity, which polypeptides are specifically selected from the group consisting of enzymes, antibodies or parts thereof, cytokines, vaccine antigens, growth factors or peptides. If genes are modified, which encode a non-coding sequence or an amino acid sequence as part of a polypeptide having a biological activity, also called “partial genes”, it may be preferred that an assembly of such partial genes has functional features, e.g. encodes a polypeptide having a biological activity. Preferably a number of different genes, e.g. different partial genes, at a size ranging from 3 bp to 20.000 bp, specifically at least 100 bp, preferably from 300 bp to 20.000 bp, specifically up to 10.000 bp, are recombined, which number of different genes of is at least 2, more specifically at least 3, 4, 5, 6, 7, 8, 9, or at least 10 to produce a recombined gene sequence that is non-coding or encoding a recombinant polypeptide, e.g. having a biological activity, which is advantageously modulated, e.g. having an increased biological activity. The term “biological activity” as used in this regard specifically refers to an enzymatic activity, such as an activity that converts a particular substrate into a particular product. Preferred genes as diversified according to the invention are coding for multi-chain polypeptides.

According to a particular embodiment of the invention there is provided a method of cell display of gene variants, comprising creating a variety of gene mosaics in cells using the method according to the invention, and displaying said variety on the surface of said cells to obtain a library of mosaics.

The library obtainable by such preferred display specifically comprises a high percentage of gene mosaics within a functional open reading frame (ORF), preferably at least 80%.

A library according to the invention specifically may be in any suitable form, specifically a biological library comprising a variety of organisms containing the gene variants. The biological library according to the invention may be contained in and/or specifically expressed by a population of organisms to create a repertoire of organisms, wherein individual organisms include at least one library member.

According to a specific aspect of the invention there is further provided an organism that comprises a gene variant from such a library, e.g. an organism selected from a repertoire of organisms. The organism as provided according to the invention may be used to express a gene expression product in a suitable expression system, e.g. as a production host cell.

### FIGURES

FIG. 1: Non-meiotic in vivo recombination

The homeologous genes A and B (homology of less than 99.5%) were recombined. As the marker sequences and the flanking target sequences are not homologous, recombination/assembly only occurred between genes A and B. As a consequence the hybrid/mosaic DNA contained recombined gene A and B, two markers and both flanking target sequences. The gene mosaic is integrated into the target locus on a target chromosome. Clones that have integrated the entire construct grew on appropriate media which is selective for both markers.

T 5′ and T 3′ correspond to the target sequences (homology of less than 99.5%) on the yeast genome (ca. 400 bp) addressing the homologous integration onto the chromosome site. M1 and M2 are the flanking markers for the double selection. Gene A and Gene B are related homeologous versions with a given degree of homology (less than 99.5%). Overlapping sequences correspond to the entire ORFs of both genes. After assembly by homeologous recombination in a MMR deficient yeast transformant, the double selection permits the isolation of recombinants.

FIG. 2: Recombination and Assembly of DNA by homeologous recombination

This figure shows a schematic presentation of a specific embodiment, wherein the cell is co-transformed with at least two genes, here DNA fragments A and B, which have homology of less than 99.5% on their overlapping fraction of 80 bp. Each DNA fragment was flanked by one selection marker.

Fragment A contained a flanking target sequence that corresponds to the 5′ end correct integration site on the chromosome and a hybridizing region that overlaps with fragment B, fragment B contained the flanking target sequence that corresponds to the 3′ integration site and a hybridizing region that overlaps with fragment A. Mismatch deficient yeast cells were transformed with the resulting fragments. The resulting transformants were plated on a medium, which is selective for both markers. Clones that can be selected for both markers were isolated, and the integrity of the assembled/integrated cluster, as well as the ORF's reconstitution of genes A and B were verified by molecular analysis of genomic DNA of selected recombinants.

T 5′ and T 3′ correspond to the target sequences (homology of less than 99.5%) on the yeast genome (ca. 400 bp) addressing the homologous integration onto the chromosome site. M1 and M2 are the flanking markers for the double selection. DNA fragments A and B can be either assembled to one gene, which can be traceable such as GFP, or can represent two genes which are assembled by this method. Overlapping sequences of all genes have homology of less than 99.5% (120 bp), permitting the reconstitution of the ORFs after assembly by homeologous recombination. Double selection permits the recombinant isolation and serves as primary verification of assembly.

FIG. 3: Recombination and Assembly of genes A, B and C

This figure shows the co-transformation of a further gene C, which has a sequence hybridizing with a flanking sequence of genes A and/or B to obtain assembly of said gene C to genes A and B.

T 5′ and T 3′ correspond to the target sequences (homology of less than 99.5%) on the yeast genome (ca. 400 bp) addressing the homologous integration onto the chromosome site. M1 and M2 are the flanking markers for the double selection. Gene A, Gene B and Gene C are related homeologous versions with a given degree of homology (less than 99.5%). Overlapping sequences correspond to the 5′ part and the 3′ part of the genes. The Gene B connects the flanking fragments and a new ORF ABC is reconstituted by sequence similarity. After assembly by homeologous recombination in a MMR deficient yeast transformant, the double selection permits the isolation of recombinants.

FIG. 4: Oxa recombination substrates

The four genes encode variants of the β-lactamase enzyme. They are related versions with a different degree of homology at the DNA level (from 95% to 47%). The sources of the parental gene sequences are Pseudomonas aeruginosa for OXA11 and OXA5 and Escherichia coli for OXA7 and Oxa1. The upper panel shows the schematic annealing of the gene's ORFs, with a dendrogramme generated after the alignment. The gene sizes are appr. 800 bp. ATG and TAA means start and stop codons. The bottom table shows the percentage of sequence similarity between the four genes at DNA level. For DNA sequences of each gene see FIG. 7.

FIG. 5: Sequences of gene and protein mosaics OXA11/OXA7 (SEQ ID NOs 1-14)

Nucleotide sequences of OXA7 origin are bold and underlined, mutation nucleotide sequences are bold and italic.

Clones were isolated by double selection and DNA used for amplification and sequencing. Only clearly readable sequences of both strands were used. Resulting chromatograms were aligned with a Clustal-like program.

FIG. 6: Sequences of gene and protein mosaics OXA11/OXA5 (SEQ ID NOs 15-38) and OXA11/OXA1 (SEQ ID NOs 67-70)

Nucleotide sequences of OXA5 or OXA1 origin are bold and underlined, mutation nucleotide sequences are bold and italic.

Clones were isolated by double selection and DNA used for amplification and sequencing. Only clearly readable sequences of both strands were used. Resulting chromatograms were aligned with a Clustal-like program.

FIG. 7: Sequences of parental genes OXA11, OXA7 and OXA5 and OXA1 (SEQ ID NOs 39-41 and SEQ ID NO 66)

FIG. 8: Sequences of clones comprising complex mosaic genes, corresponding to homeologous assembly OXA11/OXA5/OXA7

Sequences clones and results of respective protein annealing: FIG. 8a) OUL3-05-II (SEQ ID NOs 42 and 43), FIG. 8b) OUL3-05-III (SEQ ID NOs 44 and 45), FIG. 8c) OUL3-05-IV (SEQ ID NOs 46 and 47), FIG. 8d) OUL3-05-IX (SEQ ID NOs 48 and 49) and FIG. 8e) OUL3-05-X (SEQ ID NOs 50 and 51) of OXA11/OXA5/OXA7.

Nucleotide sequences of OXA 5 are bold and those corresponding to OXA 7 are underlined. Non bolded, non underlined sequences correspond to OXA 11.

FIG. 9: Sequences of ADH1 genes of Kluyveromyces lactis, Saccharomyces cerevisiae and recombinant sequences

Nucleotide sequences of Kluyveromyces lactis origin are underlined.

FIG. 9a): (SEQ ID NOs 52) ADH Kluyveromyces, FIG. 9b): (SEQ ID NOs 53) Saccharomyces, FIG. 9c): (SEQ ID NOs 54) clone A02, FIG. 9d): (SEQ ID NOs 55) A03, FIG. 9e): (SEQ ID NOs 56) A05, FIG. 9f): (SEQ ID NOs 57) A06, FIG. 9g): (SEQ ID NOs 58) A10, FIG. 9h): (SEQ ID NOs 59) A11.

FIG. 10: Sequences of clones comprising complex mosaic genes, corresponding to homeologous assembly OXA11/OXA5/OXA7 in DNA repair deficient strain of Saccharomyces cerevisiae.

Sequences show multiple cross-overs, even with genes having a homology of less than 50%. SEQ ID 60: OUL-Y00-I (DNA), SEQ ID 61: OUL-Y00-I (Protein), SEQ ID 62: OUL-Y00-IV (DNA), SEQ ID 63: OUL-Y00-IV (Protein), SEQ ID 64: OUL14-15 (DNA), SEQ ID 65: OUL14-15 (Protein), SEQ ID NO 69: OUL-Y00-15 (DNA), SEQ ID NO 70: OUL-Y00-15 (Protein).

### DETAILED DESCRIPTION OF THE INVENTION

Therefore, the present invention relates to a novel and highly efficient method for in vivo recombination of homeologous DNA sequences, i.e. similar, but not identical sequences. Hereinafter the term homologous recombination, sometimes called homeologous recombination when homeologous sequences are recombined, refers to the recombination of sequences having a certain homology, which may or may not be identical. Unlike the conventional cloning approach that relies on site-specific digestion and ligation, homologous recombination aligns complementary sequences and enables the exchange between fragments. Recombinant mosaic genes, also called hybrid genes, are generated in the cell through hybridization of sequences having mismatched bases. By such an inventive mutagenesis method it is possible to easily create a diversity for suitable selections and redesign of polypeptides of interest in a time efficient manner.

Specifically, the invention enables the first time the effective recombination and mosaic formation, diversification and assembly of diverse genes in a single step procedure, by employing the functional system of in vivo recombination.

The term “single step procedure” means that several process steps of engineering recombinants, like transformation of cells with a gene, the recombination of genes, generation of a mosaic gene and integration of a gene into the target genome, are technically performed in one method step. Thus, there would be no need of in vitro recombination of DNA carriers prior to in vivo recombination, or any repeating cycles of process steps, including those that employ meiosis. Advantageously, the use of meiotic yeast cells can be avoided.

The single step procedure according to the invention may even include the expression of such engineered recombinants by a host at the same time. Thereby no further manipulation would be necessary to obtain an expression product.

The term “gene mosaic” according to the invention means the combination of at least two different genes with at least one cross-over event. Specifically such a cross-over provides for the combination or mixing of DNA sequences. A gene mosaic may be created by intragenic mixing of gene(s), an intragenic gene mosaic, and/or gene assembly, optionally assembly of genes with both, intragenic and intergenic cross over(s) or gene mosaic(s).

The term “cross-over” refers to recombination between genes at a site where two DNA strands can exchange genetic information, i.e. at least one nucleotide. The crossover process leads to offspring mosaic genes having different combinations of genes or sequences originating from the parent genes.

Alternatively, other repair mechanisms may be provided, which are not based on cross-over, e.g. nucleotide excision repair or non homologous end joining mechanisms comprising the recognition of incorrect nucleotides, excision and/or replacement after junction of strands.

The term “flanking target sequence” refers to regions of a nucleotide sequence that are complementary to the target of interest, such as a genomic target integration site, including a site of the gene(s) A and/or other gene(s) to be recombined, linear polynucleotides, linear or circular plasmids YAC's and the like. Due to a specific degree of complementation or homology, the flanking target sequence may hybridize with and integrate gene(s) into the target integration site.

The term “genome” of a cell refers to the entirety of an organism's hereditary information, represented by genes and non-coding sequences of DNA, either chromosomal or non-chromosomal genetic elements such as, linear polynucleotides, e.g. including the gene A and/or the other gene(s) to be recombined, viruses, self replicating carriers and vectors, plasmids, and transposable elements, including artificial chromosomes and the like. Artificial chromosomes are linear or circular DNA molecules that contain all the sequences necessary for stable maintenance upon introduction in a cell, where they behave similar to natural chromosomes and therefore are considered as part of the genome.

The term “homology” indicates that two or more nucleotide sequences have (to a certain degree, up to 100%) the same or conserved base pairs at a corresponding position. A homologous sequence, also called complementary, corresponding or matching sequence, as used according to the invention preferably is hybridising with the homologous counterpart sequence, e.g. has at least 30% sequence identity, but less than 99.5% sequence identity, possibly less than 95%, less than 90%, less than 85% or less than 80%, even less than 70%, less than 60% or less than 50%, with a respective complementary sequence, with regard to a full-length native DNA sequence or a segment of a DNA sequence as disclosed herein. The sequence identity is understood to refer to identical or complementary sequences. The percent sequence identity is herein also called percent homology. Preferably, a certain homologous sequence as used herein will have at least about 30% nucleotide sequence identity, always including corresponding or complementary identity, preferably at least about 40% identity, more preferably at least about 50% identity, more preferably at least about 60% identity, more preferably at least about 70% identity, more preferably at least about 80% identity, more preferably at least about 90% identity, more preferably at least about 95% identity. Preferred ranges with upper and lower limits as cited above are within the range of 30% and 99.5% corresponding sequence identity. As used herein, the degree of identity or homology always refers to the identical or complementary nucleotide sequences.

Genes of a gene family present in different species are also called “homologues”. Preferred DNA repair genes suitable knocked-out are homologues of the specific DNA repair genes as described herein, e.g. homologues of the RAD genes or genes of the RECQ family of a variety of different eukaryotic cells, including the homologues in the preferred yeast strains. Such homologues are well-known and can be found in many eukaryotic species, which differ from each other, e.g. in the presence or absence of certain domains, and the length and sequence of the non-conserved regions.

“Percent (%) identity” with respect to the nucleotide sequence of a gene is defined as the percentage of nucleotides in a candidate DNA sequence that is identical with the nucleotides in the DNA sequence or its corresponding or complementary sequence, after aligning the sequence and introducing gaps, if necessary, to achieve the maximum percent sequence identity, and not considering any conservative substitutions as part of the sequence identity. Alignment for purposes of determining percent nucleotide sequence identity can be achieved in various ways that are within the skill in the art, for instance, using publicly available computer software. Those skilled in the art can determine appropriate parameters for measuring alignment, including any algorithms needed to achieve maximal alignment over the full length of the sequences being compared.

The term “anchoring” means the binding of a gene or gene mosaic to an integration sequence through a segment called “anchoring sequence” with partial or complete sequence homology, to enable the integration of such gene or gene mosaic into the integration site of a genome. Specifically the anchoring sequence can be a flanking target region homologous or partially homologous to an integration site of a genomic sequence. The preferred anchoring sequence has preferably at least about 70% sequence homology to a target integration site, more preferably at least 80%, 90%, 95% up to 99.55% or complete match with the hybridizing section of the genome.

The integration site may suitably be a defined locus on the host genome, where a high frequency of recombination events would occur. A preferred locus is, for example, the BUD31-HCM1 locus on chromosome III of S. cerevisiae. In general, any further loci on yeast chromosomes that show recombination at high frequencies but no change of cellular viability is preferred.

The term “expression” or “expression system” or “expression cassette” refers to nucleic acid molecules containing a desired coding sequence and control sequences in operable linkage, so that hosts transformed or transfected with these sequences are capable of producing the encoded proteins. In order to effect transformation, the expression system may be included on a vector; however, the relevant DNA may then also be integrated into the host chromosome.

The term “gene” shall also include DNA fragments of a gene, in particular those that are partial genes. A fragment can also contain several open reading frames, either repeats of the same ORF or different ORFs. The term shall specifically include nucleotide sequences, which are non-coding, e.g. untranscribed or untranslated sequences, or encoding polypeptides, in whole or in part.

The term “gene A” as used according to the invention shall mean any nucleotide sequence of a non-coding sequence or a sequence encoding a polypeptide or polypeptides of interest. Gene A is characterized by being presented in the framework of a genetic construct, such as an expression cassette, a linear polynucleotide, a plasmid or vector, which preferably incorporates at least a marker sequence and has a single flanking target sequence, either at the 5′ end or 3′ end of gene A or the genetic construct. In the method according to the invention the gene A is typically a first gene in a series of genes to be recombined for gene mosaic formation. Gene A is homologous to another gene to be recombined, which is eventually either a variant of gene A, or any of genes B, C, D, E, F, G, H, etc., as the case may be. Thereby only one flanking target sequence per gene A is typically provided for the maximum fidelity purpose. Variants of gene A are called gene A1, A2, A3, etc., which have sequence homology to a certain extent, and optionally similar functional features. The term “at least one gene A” shall mean at least gene A and optionally variants of gene A.

The term “gene B” as used according to the invention shall mean any nucleotide sequence of a non-coding sequence or a sequence encoding a polypeptide or polypeptides of interest, which is chosen for gene mosaic formation with another gene to be recombined, which is eventually either a gene A, a variant of gene B, or any of genes C, D, E, F, G, H, etc., as the case may be. Gene B is homologous to gene A or the other genes to a certain extent to enable mosaic formation with gene A or the other genes to be recombined. In the method according to the invention the gene B is typically the final gene in a series of genes to be recombined for gene mosaic formation. Gene B may be an integral part of the cell genome, or presented in the framework of a genetic construct, such as an expression cassette, a linear polynucleotide, a plasmid or vector, which preferably incorporates at least a marker sequence and has a single flanking target sequence, either at the 5′ end or 3′ end of gene B or the genetic construct, as a counterpart of the flanking target sequence of gene A, meaning at the opposite end of the gene. If the flanking target sequence of gene A is at the 5′ end of gene A, then the gene B would typically have its flanking target sequence on the 3′ end and vice versa. Thereby only one flanking target sequence per gene B is typically provided for the maximum fidelity purpose. Gene B may be a variant of gene A. Variants of gene B are called gene B1, B2, B3, etc., which have sequence homology to a certain extent, and optionally similar functional features. The term “at least one gene B” shall mean at least gene B and optionally variants of gene B.

The term “gene C” as used according to the invention shall mean any nucleotide sequence of a non-coding sequence or a sequence encoding a polypeptide of interest. Gene C is characterized by being presented in the framework of a genetic construct, such as an expression cassette, a linear polynucleotide, a plasmid or vector, which optionally incorporates a marker sequence, and further characterised by a segment of its nucleotide sequence that is homologous to a sequence of gene A and/or gene B, a variant of gene C or eventually other genes D, E, F, G, H, etc, as the case may be. Gene C preferably has a single flanking target sequence, either at the 5′ end or 3′ end of gene C, or a flanking target sequence on both sides. Thereby gene C may partially or completely hybridize with gene A and/or the other genes to recombine, link and assemble the genes. In the method according to the invention the gene C is typically the second gene following gene A in a series of genes to be recombined for gene mosaic formation. Variants of gene C are called C1, C2, C3, etc, which have sequence homology to a certain extent, and optionally similar functional features.

A further gene D may be additionally recombined and assembled through hybridization of its nucleotide sequence or a segment of its nucleotide sequence that is homologous to a sequence of gene C, a variant of gene D or eventually other genes A, B, E, F, G, H, etc, as the case may be to provide the respective recombination and linkage. Gene D preferably has a single flanking target sequence, either at the 5′ end or 3′ end of gene D, or a flanking target sequence on both sides. In the method according to the invention the gene D is typically the next gene following gene C in a series of genes to be recombined for gene mosaic formation. Variants of gene D are called D1, D2, D3, etc, which have sequence homology to a certain extent, and optionally similar functional features.

A further gene E may be additionally recombined and assembled through a segment of its nucleotide sequence that is homologous to a sequence of gene D, a variant of gene E or eventually other genes A, B, C, F, G, H, etc, as the case may be to provide the respective recombination and linkage. Gene E preferably has a single flanking target sequence, either at the 5′ end or 3′ end of gene E, or a flanking target sequence on both sides. In the method according to the invention the gene E is typically the next gene following gene D in a series of genes to be recombined for gene mosaic formation. Variants of gene E are called E1, E2, E3, etc, which have sequence homology to a certain extent, and optionally similar functional features.

Further genes F, G, H, etc. may be used accordingly. The series of further genes is understood not to be limited by the number of alphabetical letters. The final chain of genes of interest would be obtained through linkage to the genes A and B to obtain the gene assembly at the integration site of the genome. The so assembled genes of interest may be operably linked to support the expression of the corresponding polypeptides of interest and metabolites, respectively. A specific method of assembly employs the combination of cassettes by in vivo recombination to assemble even a large number of DNA fragments to obtain desired DNA molecules of substantial size. Cassettes representing overlapping sequences are suitably designed to cover the entire desired sequence. In one embodiment the preferred overlaps are at least about 5 bp, preferably at least about 10 bp. In other embodiments, the overlaps may be at least 15, preferably at least 20 up to 1.000 bp.

In one preferred embodiment, some of the cassettes are designed to contain marker sequences that allow for identification. Typically marker sequences are located at sites that tolerate transposon insertions so as to minimize biological effects on the final desired nucleic acid sequence.

In a specific embodiment the host cell is capable of recombining or assembling even a large number of genes or DNA fragments of nucleic acids with overlapping sequences, e.g. at least 2, preferably at least 3, 4, 5, 6, 7, 8, 9, more preferably at least 10 genes or nucleic acid fragments in the host cell by co-transformation with a mixture of said genes or fragments and culturing said host to which the recombined or assembled sequences are transferred.

The genes or DNA fragments to be used according to the invention, either as a whole gene or in part, can either be double-stranded or single stranded. The double-stranded nucleic acid sequences are generally 300-20.000 base pairs and the single stranded fragments are generally shorter and can range from 40 to 10.000 nucleotides. For example, assemblies of as much as 2 Mb up to 500 Mb could be assembled in yeast.

Genomic sequences from a number of organisms are publicly available and can be used with the method according to the invention. These genomic sequences preferably include information obtained from different strains of the host cell or different species to provide homologous sequences having a specific diversity.

The initial genes used as substrates for recombination are a usually a collection of polynucleotides comprising variant forms of a gene. The variant forms show substantial sequence identity to each other sufficient to allow homologous recombination between substrates. The diversity between the polynucleotides can be natural, e.g., allelic or species variants, induced, e.g. error-prone PCR or error-prone recursive sequence recombination, or the result of in vitro recombination. Diversity can also result from resynthesizing genes encoding natural proteins with alternative codon usage. There should be at least sufficient diversity between substrates that recombination can generate more diverse products than there are starting materials. There must be at least two substrates differing in at least one or more positions. The degree of diversity depends on the length of the substrate being recombined and the extent of the functional change to be evolved. Diversity up to 69% of positions is typical.

According to the inventive method it is preferred that the genes A, B, C and further genes share a homology of at least 30% at least at a specific segment designed for hybridization, which would include the full-length gene. The preferred homology percentage is at least 40%, more preferred at least 50%, more preferred at least 60%, more preferred at least 70%, more preferred at least 80%, more preferred at least 90%, even more preferred at least 95% up to less than 99.5%.

According to the invention a gene mosaic is specifically generated wherein genes are recombined that have a certain homology, such as a homology of at least 30%, and at least one intragenic gene mosaic is generated. Thereby genes, which are e.g. gene variants, may be recombined.

According to a specific embodiment, a gene mosaic is generated wherein genes are assembled. In this case the genes may have homology or partial homology within a specific region to be recombined, i.e. the overlap, or even no homology. An overlap of at least 3 bp is preferred. Where there is no overlap, the genes are assembled to align the 3′ end of one gene to the 5′ end of another gene.

Thereby genes, which encode sequences or proteins with different functions, e.g. proteins that participate in a metabolic pathway of a microorganism, may be preferably assembled.

The term “assembly” as used herein shall specifically refer to aligning and optionally merging nucleotide sequences in order to create a construct of genes that operate together in order to provide for linked activities or processes. Thus, an assembly of genes is herein understood as a series of genes (which “gene” term is herein always understood as encompassing non-coding sequences, partial genes or genes, e.g. of at least 3 up to 20,000 bp), or a string of genes, such as an alignment of genes, irrespective of the order. An assembly of the invention specifically provides for an intergenic gene mosaic. Preferably, the assembly additionally provides for at least one intragenic gene mosaic, e.g. through the use of gene variants in addition to the various different genes to provide both, the intragenic and the intergenic gene mosaic by the method of the invention.

In many cases it may be desirable simply to assemble, e.g. to string together and optionally mix such genes with gene variants, to diversify larger genes, e.g. members of an individual metabolic pathway or to assemble multiplicities of metabolic pathways according to this method. Metabolic pathways, which do not exist in nature, can be constructed in this manner. Thus, enzymes which are present in one organism that operate on a desired substrate produced by a different organism lacking such a downstream enzyme, can be encoded in the same organism by virtue of constructing the assembly of genes or partial genes to obtain recombined enzymes. Multiple enzymes can thus be included to construct complex metabolic pathways. This is advantageous if a cluster of polypeptides or partial polypeptides shall be arranged according to their biochemical function within the pathway. Exemplary gene pathways of interest are encoding enzymes for the synthesis of secondary metabolites of industrial interest, such as flavonols, macrolides, polyketides, etc.

In addition, combinatorial libraries can be prepared by mixing fragments, where one or more of the fragments are supplied with the same hybridizing sequences, but different intervening sequences encoding enzymes or other proteins.

Genetic pathways can be constructed in a combinatorial fashion such that each member in the combinatorial library has a different combination of gene variants. For example, a combinatorial library of variants can be constructed from individual DNA elements, where different fragments are recombined and assembled and wherein each of the different fragments has several variants. The recombination and assembly of a metabolic pathway may not need the presence of a marker sequence to prove the successful engineering. The expression of a metabolite in a desired way would already be indicative for the working example. The successful recombination and assembly of the metabolic pathway may, for example, be determined by the detection of the secondary metabolite in the cell culture medium.

Eukaryotic host cells are contemplated for use with the disclosed method, including yeast host cells, such as S. cerevisiae, insect host cells, such as Spodooptera frugiperda or human host cells, such as HeLa and Jurkat.

Preferred host cells are haploid cells, such as from Candida sp, Pichia sp and Saccharomyces sp.

The inventive method would not use the sexual cycle or meiotic recombination. DNA fragments can be transformed into haploid cells. The transformants can be immediately streaked out on selective plates. The recombinants would then be isolated by PCR or other means, like gap repair.

The inventive process is conducted in any eukaryotic cell with knock-outs of DNA repair genes, preferably those with deficient homologues of RAD1 and RECQ genes.

Specifically, the knock-out of DNA repair genes may be though deletion of the genes, either in whole or in part, mutation of such genes, including deletions, insertions and/or substitutions, or any other strategy that transiently or permanently impairs the DNA repair, including the mutation of a gene involved in DNA repair, treatment with UV light, treatment with chemicals, such as 2-aminopurine, inducible expression or repression of a gene involved in the DNA repair, for example, via regulatable promoters, which would allow for a transient inactivation and activation.

Bacterial DNA repair systems have been extensively investigated. In other systems, such as yeast, several genes have been identified whose products share homology with the bacterial DNA repair system, e.g. referring to analogues of RAD1 or RECQ.

In eukaryotic cells, RECQ DNA helicases comprise a family of proteins required for genome stability and resistance to DNA-damaging agents. The yeasts Saccharomyces cerevisiae and Schizosaccharomyces pombe each contain a single RECQ helicase, Sgs1 and Rqh1, respectively. Mutations in SGS1 result in increased rates of recombination, impaired sporulation, and an increased sensitivity to DNA-damaging agents. The recovery from DNA synthesis arrest is commonly known as a conserved function of RECQ DNA helicases. It was therefore surprising that a eukaryotic strain with a knock-out of such genes could be used as a host for in vivo recombination to provide gene mosaics according to the invention, without significant impairment of the cells.

Examples for preferred DNA repair deficient cells are specific yeast cells, such as S. cerevisiae strains with deletions of respective genes such as SGS1. Exemplary host cells are commercially available, e.g. the SGS1 deleted strain (Acc. No Y00775, Euroscarf Frankfurt).

The method according to the invention mainly employs marker assisted selection of a successful recombination product. The use of tools such as molecular markers or DNA fingerprinting can map the genes of interest. This allows screening of a large repertoire of cells to obtain a selection of cells that possess the trait of interest. The screening is based on the presence or absence of a certain gene.

The term “selection marker” as used according to the invention refers to protein-encoding or non-coding DNA sequences with provides for a mark upon successful integration. Specifically, the protein-encoding marker sequences are selected from the group of nutritional markers, pigment markers, antibiotic resistance markers, antibiotic sensitivity markers, fluorescent markers, knock-in markers, activator/binding domain markers and dominant recessive markers, colorimetric markers, and sequences encoding different subunits of an enzyme, which functions only if two or more subunits are expressed in the same cell. The term shall also refer to a traceable gene to be recombined that provides for the direct determination of the gene mosaic, without the need to use separate marker sequences.

A “nutritional marker” is a marker sequence that encodes a gene product which can compensate an auxotrophy of the cell and thus confer prototrophy on that auxotrophic cell. According to the present invention the term “auxotrophy” means that the cell must be grown in medium containing an essential nutrient that cannot be produced by the auxotrophic cell itself. The gene product of the nutritional marker gene promotes the synthesis of this essential nutrient missing in the auxotrophic cell. By successfully expressing the nutritional marker gene it is then not necessary to add this essential nutrient to the cultivation medium in which the cell is grown.

Preferred marker sequences are URA3, LEU2, CAN1, CYH2, TRP1, ADE1 and MET5.

A gene coding for a “pigment marker” is encoding a gene product, which is involved in the synthesis of a pigment which upon expression can stain the cell. Thereby rapid phenotypical detection of cells successfully expressing pigment markers is provided.

An “antibiotic resistance marker” is a gene encoding a gene product, which allows the cell to grow in the presence of antibiotics at a concentration where cells not expressing said product cannot grow.

An “antibiotic sensitivity marker” is a marker gene, wherein the gene product inhibits the growth of cells expressing said marker in the presence of an antibiotic.

A “knock-in” marker is understood as a nucleotide sequence that represents a missing link to a knock-out cell, thus causing the cell to grow upon successful recombination and operation. A knock-out cell is a genetically engineered cell, in which one or more genes have been turned off through a targeted mutation. Such missing genes may be suitably used as knock-in markers.

A “fluorescence marker” shall mean a nucleotide sequence encoding a fluorophore that is detectable by emitting the respective fluorescence signal. Cells may easily be sorted by well-known techniques of flow cytometry on the basis of differential fluorescent labeling.

The genes as used for diversification or recombination can be non-coding sequences or sequences encoding polypeptides or protein encoding sequences or parts or fragments thereof having sufficient sequence length for successful recombination events. More specifically, said genes have a minimum length of 3 bp, preferably at least 100 bp, more preferred at least 300 bp.

The preferred gene mosaics obtained according to the invention are of at least 3, preferably up to 20,000 base pairs, a preferred range would be 300-10,000 bp; particularly preferred are large DNA sequences of at least 500 bp or at least 1,000 bp.

Specifically preferred are gene mosaics that are characterized by at least 3 cross-over events per 700 base pairs, preferably at least 4 cross-overs per 700 base pairs, more preferred at least 5, 6 or 7 cross-overs per 700 base pairs or per 500 base pairs, which include the crossing of single nucleotides, or segments of at least 1, preferably at least 2, 3, 4, 5, 10, 20 up to larger nucleotide sequences.

According to the method of present invention not only odd but also an even number of recombination events can be obtained in one single recombined gene. This is a specific advantage over meiotic in vivo recombination.

Complex patterns of recombinant mosaicism can be obtained by the present method, reaching out high numbers of recombined sequence blocks of different length within one single molecule. Moreover, point-like replacement of nucleotides corresponding to one of the strand templates can be obtained as an important source of diversity respecting the frame of the open reading frames. Mosaicism and point-like exchange are not necessarily conservative at the protein level. Indeed, new amino acids with different polar properties can be generated after recombination, giving novel potential and enzymatic protein properties to the recombinant proteins derived by this method.

Preferably, the genes are protein-encoding sequences or parts of fragments thereof encoding enzymes or proteins of therapeutic or industrial applications. In the following the term “polypeptides” shall include peptides of interest having preferably at least 2 amino acids, preferably at least 3 polypeptides and proteins. The polypeptides of interest preferably are selected, but not limited to enzymes, members of the immunoglobulin superfamily, such as antibodies and antibody domains or fragments, cytokines, vaccine antigens, growth factors and peptides.

Enzymatic catalysts are suitably used in many industrial processes because of their high selectivity. Preferred enzymes as used for diversification according to the invention include proteolytic enzymes, such as subtilisins; cellulolytic enzymes, such as cell-wall loosening enzymes as used in the pulp and paper industry, endoglucanase, amylosucrase, aldolase, sugar kinase, cellulose, amylase, xylanase, glucose dehydrogenase and beta-glucosidase, laccase; lipases as used in the synthesis of fine chemicals, agrochemicals and pharmaceuticals; esterases, e.g. for the production of biofuel. A preferred example of enzyme improvement is the production of an alcohol dehydrogenase with improved thermostability.

It can be shown that even genes encoding multichain polypeptides with complex structures and folds can be recombined and assembled. Preferred examples are members of the immunoglobulin superfamily, among them immunoglobulins and polypeptides sharing structural features with immunoglobulins possessing a domain known as an immunoglobulin domain or fold, including cell surface antigen receptors, co-receptors and co-stimulatory molecules of the immune system, molecules involved in antigen presentation to lymphocytes, cell adhesion molecules, certain cytokine receptors and intracellular muscle proteins. Preferably antibodies or antibody fragments, such as Fab, Fv or scFv are recombined and assembled.

Alternatively, the mosaic genes can also be non-protein encoding sequences, like for example sequences which are involved in the regulation of the expression of a protein-encoding sequence, even regulatory sequences as short and long non coding RNAs. These can be but are not limited to promoter sequences, intron sequences, sequences coding for polyadenylation signals.

In a preferred embodiment of the invention the assembly of a mosaic gene, its recombination with a host genome, and further the expression of the mosaic gene to produce a recombinant polypeptide of interest or a metabolite of said host cell, is performed in a single step procedure.

In accordance with the present invention there may be employed conventional molecular biology, microbiology, and recombinant DNA techniques within the skill of the art. Such techniques are explained fully in the literature. See, e.g., Maniatis, Fritsch & Sambrook, “Molecular Cloning: A Laboratory Manual (1982).

For in vivo recombination, the gene to be recombined with the genome or other genes is used to transfect the host using standard transfection techniques. In a suitable embodiment DNA providing an origin of replication is included in the construct. The origin of replication may be suitably selected by the skilled person. Depending on the nature of the genes, a supplemental origin of replication may not be required if sequences are already present with the genes or genome that are operable as origins of replication themselves.

Synthetic nucleic acid sequences or cassettes and subsets may be produced in the form of linear polynucleotides, plasmids, megaplasmids, synthetic or artificial chromosomes, such as plant, bacterial, mammalian or yeast artificial chromosomes.

A cell may be transformed by exogenous or heterologous DNA when such DNA has been introduced inside the cell. The transforming DNA may or may not be integrated, i.e. covalently linked into the genome of the cell. In yeast and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA.

The diverse genes substrates may be incorporated into plasmids. The plasmids are often standard cloning vectors, e.g., bacterial multicopy plasmids. The substrates can be incorporated into the same or different plasmids. Often at least two different types of plasmid having different types of selectable markers are used to allow selection for cells containing at least two types of vector.

Plasmids containing diverse gene substrates are initially introduced into cells by any method (e.g., chemical transformation, natural competence, electroporation, biolistics, packaging into phage or viral systems). Often, the plasmids are present at or near saturating concentration (with respect to maximum transfection capacity) to increase the probability of more than one plasmid entering the same cell. The plasmids containing the various substrates can be transfected simultaneously or in multiple rounds. For example, in the latter approach cells can be transfected with a first aliquot of plasmid, transfectants selected and propagated, and then infected with a second aliquot of plasmid. Preferred plasmids are, for example, pUC and pBluscribe derivatives as pMXY9, pMXY12 and pMIX-LAM or YAC derivatives as YCp50.

The rate of evolution can be increased by allowing all gene substrates to participate in recombination. Such can be achieved by subjecting transfected cells to electroporation. The conditions for electroporation are the same as those conventionally used for introducing exogenous DNA into cells. The rate of evolution can also be increased by fusing cells to induce exchange of plasmids or chromosomes. Fusion can be induced by chemical agents, such as PEG, or viral proteins, such as influenza virus hemaglutinin, HSV-1 gB and gD. The rate of evolution can also be increased by use of mutator host cells (e.g., Mut L, S, D, T, H in bacteria, analogous mutants in yeast, and Ataxia telangiectasia human cell lines).

Cells bearing the recombined genes are subject to screening or selection for a desired function. For example, if the substrate being evolved contains a drug resistance gene, one would select for drug resistance.

Typically, in this inventive method of recombination, the final product of recombination that has acquired the desired phenotype differs from starting substrates at 0.1%-50% of positions and has evolved at a rate orders of magnitude in excess (e.g., by at least 10-fold, 100-fold, 1,000-fold, or 10,000 fold) of the rate of naturally acquired mutation. The final gene mosaic product may be transferred to another host more desirable for utilization of the shuffled DNA for production purposes.

In a preferred method according to the invention the host cell is displaying the gene mosaic on the cell surface using well-known cell display systems. By diversification through such hybridization a repertoire of gene variants is produced that can be suitably displayed to create a library of such variants.

Suitable display methods include yeast display and bacterial cell display. Particularly preferred libraries are yeast surface display libraries as used with many applications in protein engineering and library screening. Such libraries provide for the suitable selection of polypeptide variants with enhanced phenotypic properties relative to those of the wild-type polypeptide. Preferably cell-based selection methods are used, e.g. against surface-immobilized ligands. A commonly used selection technique comprises analyzing and comparing properties of the mutant polypeptide obtained from such library with properties of the wild-type polypeptide. Improved desirable properties would include a change of specificity or affinity of binding properties of a ligand polypeptide, which is capable of binding to a receptor. Polypeptide affinity maturation is a particularly preferred embodiment of the invention. Further desirable properties of a variant refer to stability, e.g. thermostability, pH stability, protease stability, solubility, yield or level of secretion of the recombinant polypeptide of interest.

A library obtained by the method according to the invention contains a high percentage of potential lead candidates of functional mosaic genes, which may be expressed in a functional ORF. The preferred library has at least 80% of the gene mosaics contained within a functional ORF, preferably at least 85%, at least 90%, even at least 95%. The library as provided according to the invention specifically is further characterized by the presence of the marker sequence indicating the high percentage of successful hybridization. According to the invention not only odd but also even numbers of mosaic patches can be obtained that increases the number of variants or library members in recombinant libraries produced by said method.

Usually libraries according to the invention comprise at least 10 variants of the gene mosaics, preferably at least 100, more preferred at least 1,000, more preferred at least 10⁴, more preferred at least 10⁵, more preferred at least 10⁶, more preferred at least 10⁷, more preferred at least 10⁸, more preferred at least 10⁹, more preferred at least 10¹⁰, more preferred at least 10¹¹, up to 10¹², even higher number are feasible.

The method according to the invention can provide a library containing at least 10² independent clones expressing functional variants of gene mosaics. According to the invention it is also provided a pool of preselected independent clones, which is e.g. affinity maturated, which pool comprises preferably at least 10, more preferably at least 100, more preferably at least 1,000, more preferably at least 10,000, even more than 100,000 independent clones. Those libraries, which contain the preselected pools, are preferred sources to select the high affinity variants according to the invention.

Libraries as used according to the invention preferably comprise at least 10² library members, more preferred at least 10³, more preferred at least 10⁴, more preferred at least 10⁵, more preferred at least 10⁶ library members, more preferred at least 10⁷, more preferred at least 10⁸, more preferred at least 10⁹, more preferred at least 10¹⁰, more preferred at least 10¹¹, up to 10¹² members of a library, preferably derived from a parent gene to engineer a new property to the corresponding polypeptide of interest.

Preferably the library is a yeast library and the yeast host cell preferably exhibits at the surface of the cell the polypeptide of interest having biological activity. Alternatively, the products are staying within the cell or are secreted out of the cell. The yeast host cell is preferably selected from the genera Saccharomyces, Pichia, Hansenula, Schizosaccharomyces, Kluyveromyces, Yarrowia and Candida. Most preferred, the host cell is Saccharomyces cerevisiae.

The examples described herein are illustrative of the present invention and are not intended to be limitations thereon. Different embodiments of the present invention have been described according to the present invention. Many modifications and variations may be made to the techniques described and illustrated herein without departing from the spirit and scope of the invention. Accordingly, it should be understood that the examples are illustrative only and are not limiting upon the scope of the invention.

### EXAMPLES

### Example 1

### Description

In our experimental set-up we use beta lactamase genes of the OXA class as substrate to be recombined. The advantage of the OXA genes lies in the fact that there are homeologous genes of different diversity (from 5-50%) available. These genes are therefore good candidates to test the limits of diversity of in vivo recombination. The genes are also easy to handle (about 800 bp length).

FIG. 4 shows the OXA recombination substrates: genes and homology

TABLE 1
Sequence identity of Oxa genes

| | Oxa 7 | Oxa 11 | Oxa 5 | Oxa 1 |
|---|---|---|---|---|
| Oxa 7 | 100% | | | |
| Oxa 11 | 95% | 100% | | |
| Oxa 5 | 77% | 78% | 100% | |
| Oxa 1 | 50% | 47% | 50% | 100% |

In the first experiment Oxa 11 was recombined with respectively Oxa 7 (95% identity), Oxa 5 (77% identity) and Oxa 1 (47% identity).

For reasons of comparison, we used yeast strain BY47 derived from a strain collection (EUROSCARF) that contains knock outs of auxotrophic (-ura3, -leu2) marker genes and deletion of msh2 or sgs1 genes. The gene defects in uracil and leucine biosynthetic pathway result in auxotrophy i.e. Uracil and Leucine have to be added to the growth media or the genes introduced by transformation.

In a first step gene fragments were designed that contain on one hand the marker URA3 and OXA11 or on the other hand OXA 5/7/1 respectively with the other marker LEU2. Adjacent to the 5′ end of the URA-OXA11 fragment a DNA fragment of about 400 bp was inserted (5′ flanking target sequence) that corresponds to the 5′ insertion site in the BUD 31 region of the yeast chromosome. At the 3′ end of the OXA 5/7/1 a DNA fragment of about 400 bp (3′ flanking target sequence) corresponding to the adjacent 3′ site on the chromosome (s. FIG. 3). All fragments were synthesized according to standard protocols at Geneart (Germany).

The synthesized fragments were amplified by PCR and used for transformation.

The URA3-OXA 11 fragment and one of the other OXA-LEU2 fragments were transformed into wild-type (diploid BY26240, Euroscarf), mismatch deficient (haploid a-mater BY06240, msh2-, Euroscarf) or RecQ DNA repair deficient (haploid a-mater BY00775, sgs1-, Euroscarf) strains. The transformation protocol was according to Gietz [Gietz, R. D. and R. A. Woods. (2002) TRANSFORMATION OF YEAST BY THE Liac/SS CARRIER DNA/PEG METHOD. Methods in Enzymology 350: 87-96]. The transformants were plated on plates containing selective media for the selection on the appropriate markers (no Uracil, Leucine). After 72 hours colonies could be observed.

TABLE 2
Number of clones obtained after transformation/selection

| | Oxa11/ | Oxa11/ | Oxa11/ | Oxa11/ |
|---|---|---|---|---|
| | Oxa11 | Oxa07 | Oxa05 | Oxa1 |
| Yeast/trafo | (1) | (2) | (3) | (4) |
| BY26240 | 10⁶ (5) | <10 | 0 | ND |
| (diploid msh+) | | | | |
| BY06240 | 5 × 10⁴ | 5 × 10³ | 10³ | ND |
| (haploid Δmsh2) | | | | |
| BY00775 | — | — | 10⁴ | 4 |
| (haploid Δsg1) | | | | |

(1) Homologous control
(2) 5% of divergence at DNA level
(3) 23% of divergence at DNA level
(4) 53% of divergence at DNA level
(5) Estimated cfu (colony forming units) number per ml of transformation mix and μg of DNA on selective media (−ura −leu).

A total of 48 colonies issued from BY06240 and 4 from BY00775 transformations were isolated and colony PCR performed (lysis and Herculase PCR based on Cha and Thilly protocol: Specificity, Efficiency and fidelity of PCR, in PCR primer: A laboratory Manual, Dieffenbach and Dveksler eds. 1995, pp 37). Different PCR reactions are performed to verify the correct insertion of the fragments into the target region. 37 clones out of 48 showed correct insertion profiles. From these 37, 31 gave clear and exploitable amplification products for sequencing. The reaction that uses two specific primers flanking the Oxa ORFs only permits the amplification of true recombinants if OXA sequences were actually assembled. Additionally, the obtained product is a correct substrate for direct sequencing. Thus, the positive amplification products were sequenced (GATC). In the case of OXA11-OXA1 recombinanation (only performed in Δsgs1 background), PCR profiles were longer than those expected.

Results of Sequencing

24 clones out of 31 (those with the clearer positive amplification signals) were sequenced. They corresponded to: homologous control Oxa11/Oxa11 (SEQ ID NO 39), homologous control Oxa07/Oxa07 (SEQ ID NO. 40), homologous control Oxa05/Oxa05 (SEQ ID NO 41). fe02 to fe06, fe09 and fell: Oxa11/Oxa07 (SEQ ID NO. 1 to SEQ ID NO. 14). fe09 and fe13, fe14, fe16 to fe24: Oxa11/Oxa5 (SEQ ID NO. 15 to SEQ ID NO. 38). OUL-Y06-8 and OUL-Y00-15, OXA11/OXA5 (SEQ ID NO. 67 and 70, respectively)

For sequencing results of all of the clones see FIGS. 5 and 6 and SEQ ID NOs 1 to 38 and 67 to 70.

For DNA annealing of Oxa11/Oxa07 clones see FIG. 5, SEQ ID NOs. 1, 3, 5, 7, 9, 11 and 13.

For DNA annealing of Oxa11/Oxa05 clones see FIG. 6, SEQ ID NOs. 15, 17, 19, 21, 23, 25, 27, 29, 31, 33, 35, and 37)

For DNA annealing of Oxa11/Oxa1 clones see FIG. 6, SEQ ID NOs 67 and 69. For protein sequence of OXA11/Oxa07 see FIG. 5, SEQ ID NOs. 2, 4, 6, 8, 10, 12 and 14.

For protein sequence of Oxa11/Oxa05 see FIG. 6, SEQ ID NOs. 16, 18, 20, 22, 24, 26, 28, 30, 32, 34, 36 and 38.

For protein sequence of Oxa11/Oxa01 see FIG. 6, SEQ ID NOs 68 and 70.

### Example 2

### Description

A further comparative example refers to generating libraries of complex mosaic genes. A skilled person will be able to apply such example to the present invention employing a eukaryotic strain with knock-outs of DNA repair genes.

Three different but related gene sequences were assembled and recombined. As in example 1, OXA gene sequences were used for their assembly in MMR deficient yeast (for OXA gene identity see FIG. 4). As showed in FIG. 3, the principle of mosaic generation is based on the usage of respectively truncated sequences of OXA 11 (gene A) and OXA 7 (gene B) that hybridize with the entire ORF of OXA 5 (gene C). Thus, only assembled and integrated cassettes A-B-C sharing the auxotrophic markers will be selected after transformation.

As in example 1 we used yeast strain BY47 derived from a strain collection (EUROSCARF) that contains knock outs of auxotrophic (-ura3, -leu2) marker genes and a deletion of msh2. The gene defects in uracil and leucine biosynthetic pathway result in auxotrophy: i.e. Uracil and Leucine have to be added to the growth media.

New gene fragments containing truncated genes A and B were obtained by specific PCR from the already described fragments in the example 1: URA-Oxa11 (reverse primer annealing on nucleotides 386-406 of OXA11 ORF) and OXA7-Leu (forward primer annealing on nucleotides 421-441 of OXA 7 ORF). The entire ORF of OXA 5 gene was obtained by PCR from fragment OXA5-Leu. The fragment END-Leu was used as in example 1. Purified PCR fragments were used for transformation.

The transformation protocol was according to Gietz [Gietz, R. D. and R. A. Woods. (2002) Transformation of Yeast by the Liac/SS Carrier DNA/PEG Method. Methods in Enzymology 350: 87-96]. The transformants were plated on plates containing selective media for the selection on the appropriate markers (no Uracil, Leucine). After 72 hours colonies could be observed.

TABLE 3
Number of clones obtained after
transformation/selection

| | Oxa11/ | |
|---|---|---|
| | Oxa5/ | Oxa11/ |
| | Oxa7 | Oxa07 |
| Yeast/trafo | (1) | (2) |
| BY26240 | <10¹ (3) | ND (5) |
| (diploid | | |
| msh2+) | | |
| BY06240 | 1.4 × 10⁴ (4) | <5 |
| (haploid | | |
| Δmsh2) | | |

(1) Three OXA sequences to assemble
(2) Middle sequence OXA5 is missing (negative control)
(3) Homeologous recombination background in MMR proficient yeast
(4) Homeologous recombination background in MMR deficient yeast
(5) ND = no colony detected

A total of 8 colonies issued from BY06240 transformation were randomly isolated and colony PCR performed (lysis and Herculase PCR based on Cha and Thilly protocol: Specificity, Efficiency and fidelity of PCR, in PCR primer: A laboratory Manual, Dieffenbach and Dveksler eds. 1995, pp 37). Different PCR reactions were performed to verify the correct insertion of the fragments into the target region. 7 clones out of 8 showed correct insertion profiles. From these 7 gave clear and exploitable amplification products for sequencing. The reaction that uses two specific primers flanking the Oxa ORFs only permits the amplification of true recombinants if OXA sequences were actually assembled. Additionally, the obtained product is a correct substrate for direct sequencing. Thus, the positive amplification products were sequenced (GATC).

Results of Sequencing

7 clones out of 8 (those with the clearer positive amplification signals) were sequenced 5 exploitable sequences were obtained. They corresponded all to homeologous assembly OXA11/OXA5/OXA7 from clones OUL3-05-II, OUL3-05-III, OUL3-05-IV, OUL3-05-IX and OUL3-05-X.

For sequencing results of all of the clones and protein annealing see FIG. 8: OUL3-05-II (SEQ ID NOs 42 and 43), OUL3-05-III (SEQ ID NOs 44 and 45), OUL3-05-IV (SEQ ID NOs 46 and 47), OUL3-05-IX (SEQ ID NOs 48 and 49) and OUL3-05-X (SEQ ID NOs 50 and 51) of OXA11/OXA5/OXA7.

Discussion

This simple transformation method of mitotic DNA repair deficient cells with divergent sequences as templates for the assembly by the cell and generation of diversity by in vivo recombination has been proven (FIGS. 5, 6 and 8).

Complex patterns of recombinant mosaicism have been obtained by the method described in examplel, reaching out at least 17 patches of different length into one single molecule of 800 bp (i.e. clones fe19 (SEQ ID NO 27) and fe20 (SEQ ID NO. 28). Recombination events seem to take place all the long of the sequences.

Moreover, point-like replacement of nucleotide corresponding to one of the strand templates were observed as an important source of diversity respecting the frame of the ORFs (i.e. clones fe19 (SEQ ID NO. 27) and fe20 (SEQ ID NO. 29).

Recombination events were even observed when sequences diverged as far as 53% in transformations between OXA11 and OXA1 (i.e. clones OUL-Y06-8 (SEQ ID NO. 67) as well as mosaicism (i.e. clone OUL-Y00-15 (SEQ ID NO. 69).

In addition, this recombination method produced mosaics from more than two related genes as shown in the example 2 by using sequences from three related genes (OXA 11, OXA 7 and OXA 5) at the same time (i.e. clones OUL3-05-III and OUL3-05-IX). This is a highly efficient way to recombine regions of interest from several genes, and represents a new source of divergence based on the generation of mosaic genes libraries in vivo.

None of the recombinant clones yielded truncated protein products as verified by in silico analysis of translated DNAs (FIGS. 5, 6 and 8), except for clone OUL-Y06-8 (SEQ. ID NO. 69) in which recombination at the level of the last and first nucleotide of intervening genes gave a tandem-like recombinant conserving the stop codon of the first gene at the junction.

Only 1 clone (fel 5) out of 21 showed a parental profile (data not shown).

Mosaicism and point-like exchange are not necessarily conservative at the protein level. Indeed, new amino acids with different polar properties were generated after recombination, giving novel potential and enzymatic protein properties to the recombinant muteins (i.e. clones fe19 (SEQ ID NO. 27) and fe20 (SEQ ID NO. 29)

One very attractive trait of the recombinant generation by this approach making recombinant libraries richer is the fact that not only odd but also even number of recombination events could be obtained (i.e. fe06 (SEQ ID NO 7), fell (SEQ ID NO 13), fe13 (SEQ ID NO 17), fe19 (SEQ ID NO 27), compared to the meiotic recombination approach, by which only odd events could be represented into the library.

Some point mutations, not related to parental templates, were observed in a few numbers of sequences i.e. fe16 (SEQ ID NO 21), fe17 (SEQ ID NO 23) and OUL-Y00-15 (SEQ ID NO 23). In all those cases, the mutations didn't change the reading frame of the resulting ORFs.

### Example 3

### ADH 1

In a second example we choose an endogenous DNA as target for recombination. Alcohol dehydrogenase 1 (ADH1) is the key enzyme for the production of Ethanol in yeast Saccharomyces cerevisiae. It is of industrial interest to generate improved Adh1 variants.

The strains BY06246 from Euroscarf and W303 from Euroscarf are used for this experiment.

The Saccharomyces cerevisiae ADH1 gene is already located on chromosome XV. Therefore, introduction of only one homeologous gene is sufficient for recombination. In order to assure that recombined recombinants will not further mutate we also re-establish the mismatch repair wild-type. Therefore we additionally add a fragment containing functional MSH2 gene with its promoter and terminator regions.

As partner for somatic gene recombination we choose the Kluyveromyces thermotholerans/Lachancea thermotolerans ADH1 gene which has 82% homology with the Saccharomyces cerevisiae gene. Two fragments are designed. One fragment contains the K. thermotholerans ADH1 open reading frame. At its 3′ end a fragment containing 296 bp of the terminator region from TRP1 gene cassette comprising 283 bp of the promoter and the first 743 bp of URA3 ORF from Kluyveromyces lactis is designed. The URA3 gene product of K. lactis can complement the ura3 defect in Saccharomyces cerevisiae. The second fragment contains the last 160 bp of URA3 and 223 bp of the terminator region of URA3. This sequence is followed by 468 bp of the endogenous MSH2 promoter and the MSH2 ORF (2894 bp) and 242 bp of the TEF1 terminator. The fragment is flanked at the 3′ side by a 403 bp sequence which is identical to the of the insertion site on Chr. XV. All fragments are synthesized at Geneart.

As the 3′ end of the ADH1-URA3 fragment and the 5′ end of the URA3-MSH2 fragment are homologous the two fragments can assemble. After assembly the recombination with the Saccharomyces cerevisiae ADH1 gene and the integraton step takes place.

After transformation several clones were randomly isolated and DNA was prepared. The DNA of the ADH recombinants was sequenced. The underlined sequences are derived from the ADH Kluyveromyces lactis, the other from ADH Saccharomyces cerevisiae (see FIG. 9).

### Example 4

To test a DNA repair deficient strain for in vivo assembly and recombination capacity we used a SGS1 deleted strain (Acc. No Y00775, Euroscarf Frankfurt) and transformed it with the different recombination substrates as described in Example 1.

We detected transformants of all oxa pairs even for 47% homology. The sgs1 deletion strain was therefore able to assemble and recombine oxa genes of high divergence. Again analytical PCR was performed to identify correctly assembled and integrated clones. Oxa recombinants were analysed by sequencing. For OXA11/OXA1, PCR profiles of the recombinant sequences were larger than expected and similar to those obtained in the example 1.

TABLE 4
Number of clones obtained after transformation

| | Oxa11/ | Oxa11/ | Oxa11/ |
|---|---|---|---|
| | Oxa11 | Oxa05 | Oxa1 |
| Yeast/trafo | (1) | (2) | (3) |
| BY26240 | 10⁶ (4) | 0 | ND |
| (diploid MMR | | | |
| proficient) wild- | | | |
| type | | | |
| BY06240 | 8 × 10⁵ | 2 × 10⁴ | 6 |
| (haploid Δsgs1, | | | |
| Y00775) | | | |

(1) Homologous control
(2) 23% of divergence at DNA level
(3) 53% of divergence at DNA level
(4) Estimated cpu number per ml of transformation mix and μg of DNA on selective media (−ura −leu).

Results of Sequencing

3 clones out of 48 were sequenced and showed multiple cross-overs: two for oxa 11/5 (OUL-Y00-I and IV) and one for oxa 11/1 (OUL14-15). For sequencing results of these three clones see FIG. 10 and SEQ ID NOs: SEQ ID 60: OUL-Y00-I (DNA), SEQ ID 61: OUL-Y00-I (Protein), SEQ ID 62: OUL-Y00-IV (DNA), SEQ ID 63: OUL-Y00-IV (Protein), SEQ ID 64: OUL14-15 (DNA), SEQ ID 65: OUL14-15 (Protein).

1. A method for generating a gene mosaic by somatic in vivo recombination, comprising
a) in a single step procedure
(i) transforming a cell with at least one gene A having a sequence homology of less than 99.5% to another gene to be recombined that is an integral part of the cell genome or presented in the framework of a genetic construct,
(ii) recombining said genes,
(iii) generating a gene mosaic of the genes at an integration site of a target genome, wherein said at least one gene A has a single flanking target sequence either at the 5′ end or 3′ end anchoring to the 5′ or 3′ end of said integration site, and
b) selecting clones comprising the gene mosaic,
wherein said cell is a eukaryotic strain with a knock-out of at least one DNA repair gene.

2. The method of claim 1, wherein said DNA repair gene is completely or temporarily knocked-out, preferably by mutation, such as a deletion and/or insertion and/or substitution of one or more nucleotides.

3. The method of claim 1, wherein said DNA repair gene is selected from the group consisting of homologues of RAD1 and RECQ.

4. The method of claim 1, wherein said strain comprises a cell selected from the group consisting of fungal, yeast, plant, insect and mammalian cells.

5. The method of claim 1, wherein said strain is from a genus selected from the group consisting of Saccharomyces, Schizosaccharomyces, Saccharomyces, Candida, Kluyveromyces, Hansenula, Schizosaccaromyces, Yarrowia, Pichia, Aspergillus, Drosophila and Caenorhabditis.

6. The method of claim 5, wherein said strain is selected from group consisting of Saccharomyces cerevisiae with a knock-out of at least the SGS1 gene, Schizosaccharomyces pombe with a knock-out of at least the RQH1 gene, Drosophila melanogaster with a knock-out of at least the dmblm gene, Caenorhabditis elegans with a knock-out of at least one of F18C5.2 and TO4A11.6 genes, plants with a knock-out of at least one of AtRECQL1 to 4 and 4B genes, and mammalian cells with a knock out of at least one of BLM, WRN, RECQL, RECQL4 and RECQL5 genes.

7. The method of claim 1, wherein a selection marker is used in the gene mosaic and the clones are selected according to the presence of the selection marker.

8. The method of claim 1, wherein said another gene is part of the genome of the cell.

9. The method of claim 1, wherein the cell is co-transformed with at least one gene A and at least one gene B, wherein said single flanking target sequence of gene A is anchoring to the 5′ end of an integration site on said target genome, and wherein gene B is linked to a single flanking target sequence anchoring to the 3′ end of the integration site.

10. The method of claim 1, wherein the cell is co-transformed with at least two different genes A1 and A2 and optionally with at least two different genes B1 and B2.

11. The method of claim 1, wherein at least one further gene C is co-transformed, and wherein the gene C has a sequence hybridizing with a sequence of gene A and/or said another gene to obtain assembly of said further gene C to gene A and/or said another gene.

12. The method of claim 1, wherein said gene A and/or said another gene is coding for a polypeptide or part of a polypeptide having an activity.

13. The method of claim 1, wherein multiple genes coding for polypeptides of a biochemical pathway are recombined and assembled.

14. The method of claim 1, wherein the flanking target sequence is at least 5 bp.

15. The method of claim 1, wherein the flanking target sequence has homology in the range of 30% to 99.5% with the anchoring sequence of said integration site, preferably at least 50%.

16. The method of claim 1, wherein a selection marker is used, and wherein the selection marker is selected from the group consisting of nutrition auxotrophic markers, antibiotics resistance markers, fluorescent markers, knock-in markers, activator/binding domain markers and dominant recessive markers and colorimetric markers.

17. The method of claim 1, wherein said genes are comprised in a linear polynucleotide, a vector or a yeast artificial chromosome.

18. The method of claim 1, wherein said genes are linear polynucleotides, preferably of 300 to 20,000 bp.

19. The method of claim 1, wherein at least one clone having an intragenic gene mosaic is selected.

20. The method of claim 1, wherein at least one clone having a gene assembly and at least one intragenic gene mosaic is selected.

21. The method of claim 1, wherein gene mosaics of at least 3 up to 20,000 base pairs, preferably with at least 3 cross-over events per 700 bp are obtained.

22. The method of claim 1, wherein the genes are non-coding sequences or encoding variants of a polypeptide selected from the group consisting of enzymes, antibodies or parts thereof, cytokines, growth factors, vaccine antigens and peptides.

23. A method of cell display of gene variants, comprising creating a variety of gene mosaics in cells using the method of claim 1, and displaying said variety on the surface of said cells to obtain a library of mosaics.

24. A library of gene mosaics obtainable by the method of claim 1, wherein at least 80% of the gene mosaics are contained within a functional ORF.

25. A library according to claim 24, comprising a variety of organisms containing the gene variants.

25. An organism that comprises a gene variant from a library according to claim 24.
